(19) United States
(12) Patent Application Publication
(10) Pub. No.: US 2013/0177987 A1
(43) Pub. Date: Jul. 11, 2013

(54) METHODS FOR DEVELOPING ANTIGEN-SPECIFIC ANTIBODY-PRODUCING CELL LINES AND MONOCLONAL ANTIBODIES

(71) Applicants: Brian Schram, Madison, WI (US); Rachel Kravitz, Madison, WI (US)

(72) Inventors: Brian Schram, Madison, WI (US); Rachel Kravitz, Madison, WI (US)

(73) Assignee: NEOCLONE BIOTECHNOLOGY INTERNATIONAL, LLC, Madison, WI (US)

(21) Appl. No.: 13/531,957

(22) Filed: Jun. 25, 2012

Related U.S. Application Data
(60) Provisional application No. 61/500,973, filed on Jun. 24, 2011.

(51) Int. Cl. C12N 5/16; C12N 5/0781

(52) U.S. Cl. CPC C12N 5/163; C12N 5/0635
USPC 435/449; 435/377; 435/456; 435/355

(57) ABSTRACT

Disclosed are methods for producing class-switched, affinity-matured antibodies which include enriching an immunized cell population for GL7-positive cells and activating the enriched cells. The methods may be used to improve the efficiency of obtaining immortalized antigen-specific plasma cells or to improve the quality of molecularly cloned Ig heavy and light chains.

### CROSS-REFERENCE TO RELATED APPLICATIONS

The present application claims the benefit of priority under 35 U.S.C. 119(e) to U.S. Provisional Patent Application No. 61/500,973, filed on Jun. 24, 2011, the content of which is incorporated herein by reference in its entirety.

### FIELD

The presently disclosed methods relate generally to the field of antibody development and production. In particular, the methods relate to the field of in vitro monoclonal antibody production.

### BACKGROUND

Monoclonal antibodies (mAbs) are highly specific affinity reagents used for detecting and treating diseases. For example, mAbs may be used in localizing biomarkers in tissue, purifying biomarkers from complex substances, and measuring markers leading to diagnosis of diseases in clinical samples (e.g., cancer). The need for mAbs as affinity reagents is continually growing with the advent of multi-analyte detection platforms such as protein microarrays. The advancement of these platforms has yielded the potential for fast, high-throughput analysis of complex samples for small molecules and proteins of interest. The next few years will likely see rapid advancement in the use of these platforms for disease biomarker discovery, allowing, for example, the early-stage diagnosis of different cancers.

Despite the advancement of multi-analyte platforms and therapeutic mAbs, the overall performance and usefulness of these approaches depends on the quality of the mAbs. Successful development of antibodies requires the screening of many mAbs for affinity, specificity, cross-reactivity, immunogenicity, and platform compatibility. For a given target, dozens of mAbs may need to be screened, and thus the techniques used to generate the mAbs must be able to generate a panel of highly diverse antigen-specific (Ag-specific) mAbs. Currently, the methods for developing mAbs are limited to a few different strategies, each with limited abilities to generate clonally diverse panels of Ag-specific mAbs.

The standard method for creating mAbs to a particular antigen involves the creation of a fused cell called a “hybridoma.” A hybridoma is produced by fusing together an established tumor cell line, such as a myeloma cell line, and an antibody-producing cell (such as a B-lymphocyte) from an animal that has previously been immunized with the antigen. Antibody-producing cells typically are obtained from the animal's spleen, lymph nodes, or lymph tissue (e.g., splenocytes or lymphocytes). These fused cells or “hybrid cells” then are selected and screened to obtain a cell that produces the desired antibody (i.e., a “hybridoma”).

Hybridoma technology has significant disadvantages. Typically, one hybridoma clone may be generated per 10⁵-10⁶ splenocytes fused, thus most of the Ag-specific cells contained within the splenocyte population may be lost. In addition, many of the clones generated may not produce mAbs that recognize the antigen of interest. Furthermore, hybridoma cell lines of interest must be separated through screening and subcloning. The frequency of producing successful, Ag-specific B cell hybridomas may be on the order of one per 10⁶-10⁸ starting cells. Finally, hybridomas are polyploid and chromosomally unstable. As a result, months of in vitro culture may be required to stabilize each clone and ensure strong mAb production.

An alternative to traditional hybridoma technology is phage display. Phages are viruses that infect bacteria such as E. coli. The phage genome is replicated within the bacteria, translocated to the cytoplasm and packaged into rod-shaped particles, which are then released into the media upon bacterial lysis. The particle coats can be engineered to “display” ligands such as antibodies. Thus large phage libraries containing billions of different antibody genes can be generated, with each phage containing a single antibody gene. These libraries can be screened for binding against an antigen of interest and the desired clone selected. A major advantage of phage display is that it does not require animal immunization. However, phage display also has one primary drawback, which is antibodies developed using naïve antibody phage libraries may have affinities that are generally two to three orders of magnitude lower than those of antibodies produced using traditional fusion technology. Increasing the probability of obtaining high-affinity antibodies with phage display requires additional mutagenesis upon clone selection, greatly increasing the naïve library size, and/or generating libraries from immunized animals. Each option requires extensive development time and expense.

Plasmacytoma technology may be used as an alternative to hybridoma technology and phage display technology. Plasmacytomas are immortalized, antibody producing cells. Plasmacytomas may be obtained by infecting B cells with an immortalizing retrovirus such as the ABL-MYC retrovirus. The ABL-MYC retrovirus is a replication-defective retrovirus, which contains v-abl from the Abelson Murine Leukemia virus (Ab-MuLV) and murine c-myc. Infection by the ABL-MYC retrovirus stably transforms Ag-specific B cells into immortalized plasmacytomas that produce an antibody to a specified target antigen. Antigen-specific plasmacytomas may be obtained by infecting splenocytes from immunized mice with the ABL-MYC retrovirus and then subsequently injecting the infected splenocytes into recipient mice for plasmacytoma and ascites development. This process may provide antibodies against a wide range of antigens. However, clonal diversity may be limited by in vivo clonal selection, plasmacytoma development, and plasmacytoma propagation. For example, one disadvantage of plasmacytoma technology is that clonal diversity may be limited by in vivo ascites development, where plasmacytomas develop in the peritoneal space of the mouse, and competition between clones limits the number of different antibody cell lines that can be harvested. Ag-specific clones may be lost to other clones with more aggressive growth characteristics. Often, each recipient mouse will yield one cell line.

Molecular cloning and protein expression techniques are another way to generate monoclonal antibodies. However, these techniques typically require extensive screening after target cells of interest are identified.

Thus, new methods for obtaining antigen-specific B-lymphocytes are desirable. In particular, new methods for selection and clonal expansion of antigen-specific B-cell populations to produce stable hybridomas, plasmacytomas, and antibody-producing cell lines are desirable.

### SUMMARY

Disclosed are methods for creating antigen-specific antibody-producing cell lines. In particular, the methods may be utilized to generate antigen-specific plasmacytomas and hybridomas.

The methods may include the following steps: (a) contacting lymphocytes with an antigen to obtain immunized cells (e.g., immunized B-lymphocytes); (b) selecting the immunized cells based on expression of one or more cell surface markers or lack thereof (such as, for example, by enriching the immunized cells for cells that bind to an anti-GL7 antibody); (c) contacting the enriched population of immunized cells with an activating agent (such as, for example, the antigen, a cytokine, and/or immune cells) to obtain activated cells; and optionally (d) producing antibody from the activated cells, via molecular cloning or by immortalization.

In some embodiments, the selected, activated immunized cells obtained by the methods may be immortalized by transfecting the selected immunized cells with a viral vector that transforms the transfected cells to obtain plasmacytoma cells. In other embodiments, the selected, activated immunized cells may be immortalized by fusing the selected, activated immunized cells and myeloma cells to obtain hybridoma cells. In further embodiments the antibody genes may be molecularly cloned into expression vectors, which subsequently are transfected into cell lines for generation of antibodies. The cloned antibodies may be further assayed to ensure antigen specificity and affinity. The methods further may include culturing the immortalized antigen-specific antibody-producing cells to obtain antibodies that bind specifically to the antigen (e.g., monoclonal antibodies), or culturing cells transfected with the molecularly cloned antibody coding sequences to obtain monoclonal antibodies.

The steps of the disclosed methods may be performed in vivo or ex vivo, either entirely or in part. In some embodiments, growing may include culturing the cells in vitro or transferring the cells into a host animal for growth and/or selection in vivo. In further embodiments, one or more steps of the methods may be performed in a host animal such as a mouse (e.g., a Balb/c mouse). The host animal may be immunodeficient, such as a mouse that has a severe combined immunodeficiency mutation (i.e., a SCID mouse).

In some embodiments, the methods may include growing the selected immunized cells in the presence of an activating agent. Suitable activating agents may include cytokines as disclosed herein (e.g., IL-21) and antigens (e.g., the antigen utilized for immunizing the immunized cells as disclosed herein). In further embodiments, the selected immunized cells may be grown in the presence of the activating agent for at least one (1) day (e.g., about 1-4 days or about 2-10 days).

The cells used in the methods may be obtained from any suitable source. In some embodiments, lymphocytes may be obtained from one or more of spleen cells (e.g., splenocytes), peripheral blood leukocytes, bone marrow cells, and cord blood cells. Lymphocytes may include B-lineage lymphocytes (or “B cells”). The lymphocytes may be contacted with antigen to obtain immunized cells (e.g., immunized B lymphocytes), which subsequently are selected (e.g., by enriching for cells that bind to an anti-GL7 antibody); activated (e.g., by contacting the enriched cells with an activating agent); and immortalized (e.g., by fusion with a myeloma or by transfection with a transforming viral vector); or the antibody genes present in the selected lymphocytes may be molecularly cloned into expression vectors.

Typically, the immunized cells are selected prior to immortalization or cloning, which may include enriching the immunized cells for a sub-population of cells that express a cell surface marker. For example, the immunized cells may be selected using fluorescence-activated cell sorting (“FACS”). For example, the cells may be selected and separated into populations that express the antigen recognized by an anti-GL7 antibody to obtain one or more selected populations of immunized cells prior to immortalization. The GL7 epitope is an Neu5Ac α2-6 LacNAc-containing N-glycan. In other embodiments, the immunized cells may be selected based on expression or lack thereof of one or more cell surface markers such as CD138, CD38, CD20, CD40, CD45, CD3e, CD11b, CD19, F4/80, and CD79: in some embodiments, the immunized cells also may be selected to obtain a population of cells that is capable of being transformed at a relatively high efficiency (e.g., at least about 10%, 20%, 30%, 40%, 50%, 60%, 70%, 80%, or 90%). The immunized cells further may be selected and separated into populations based on relative expression of an antibody isotype (e.g., IgD). For example, selected populations may include B cells characterized as “germinal center cells” (e.g., GL7^{+/high}), “naïve cells” (e.g., CD138^{low}/IgD^{high}); “memory cells” (e.g., CD138^{low}/IgD^{low}); “plasmablasts” (e.g., CD138^{high}/IgD^{low}); and “plasma cells” (e.g., CD138^{high}/B220^{low}/IgD^{low}).

in some embodiments, the immunized cells further may be grown in vitro prior to immortalization or molecular cloning. For example, the immunized cells may be grown in vitro to select a population of cells that is capable of being immortalized at a relatively high efficiency (e.g., at least about 10%, 20%, 30%, 40%, 50%, 60%, 70%, 80%, or 90%). In some embodiments, the immunized cells may be grown in vitro in the presence of one or more factors that may include cytokines (e.g., interleukins (such as IL-1, IL-2, IL-4, IL-5, IL-6, IL-10, BAFF, APRIL, and IL-21), interferons, growth factors, tumor necrosis factor (TNF), cell surface ligands (e.g., CD40 ligand), antibodies (e.g., monoclonal antibodies against cell surface markers such as CD40 or IgM), Toll like receptor agonists (TLR) (e.g., Imiquimod for TLR7 and oligonucleotide (ODN)2006 for TLR9 in humans and ssRNAA40/LyoVec for TLR7 and ODN1826 for TLR9 in mice) and mitogens (e.g., pokeweed mitogen). The factor (e.g., a cytokine) may be recombinant, purified native, or unpurified native obtained from conditioned media (e.g., an unpurified cytokine from activated T cell/macrophage cultures). In further embodiments, the immunized cells may be activated with antigen in vitro prior to immortalization.

The immunized cells further may be grown in vitro in the presence of cells other than the immunized cells. For example, the immunized cells may be grown in the presence of antigen-specific T cells, dendritic cells, or macrophages. In some embodiments, the immunized cells may be grown on feeder layer cells (e.g., gamma-irradiated cells such as OP-9). The immunized cells may be grown with cells that are capable of activating the immunized cells to proliferate, differentiate, and/or secrete antibody. In some embodiments, the immunized cells are grown with a thymoma cell line such as the murine EL4 thymoma cell line, which optionally, may have been mutagenized. For example, the thymoma cell line may have been mutagenized to obtain bromo-deoxyuridine-resistant mutants (e.g., EL4-B5 cells). In some embodiments, the immunized cells are grown with macrophages (e.g., PD3188 cells or P388D1 cells) and/or T-cells. Optionally, the thymoma cells, macrophages, and/or T-cells may be stimulated or activated, for example with ultraviolet irradiation, phorbol 12-myristate 13-acetate (PMA), and/or phytohemagglutinin (PHA). Optionally, the thymoma cells, macrophages, and/or T-cells may be treated with gamma irradiation (e.g., to prevent proliferation).

In further embodiments, the immunized cells may be selected to obtain an antigen (Ag)-specific population (i.e., a population that secretes antibodies that specifically bind to the immunizing antigen). For example, the immunized cells may be selected by contacting the cells with the antigen to select an Ag-specific population. In some embodiments, the antigen is immobilized and the immunized cells are contacted with the immobilized antigen to select an Ag-specific population. In other embodiments, an Ag-specific population is selected by removing from the immunized cells those cells that are not Ag-specific, such as non-B cells and non-Ag-specific cells. For example, non-B cells and non-Ag-specific cells may be removed by contacting non-B cells and non-Ag-specific cells with labeled antibodies that bind specifically to a cell surface marker that characterizes the non-B cells or the non-Ag-specific cells.

In some embodiments, the immunized cells may be selected and activated to obtain a population of cells that subsequently is immortalized or Ig heavy and light genes molecularly cloned, where a significantly increased percentage of the immortalized cells are observed to produce antigen-specific antibodies as compared to methods utilized in the prior art. For example, the immunized cells may be selected or depleted based on the expression of a cell surface marker or the lack thereof (e.g., GL7, CD138) to obtain selected immunized cells which are subsequently immortalized or the antibody encoding fragments molecularly cloned. The immunized cells also may be grown in vitro in the presence of activating agents (e.g., cytokines such as IL-21 or Ag) prior to being immortalized.

In some embodiments of the methods for producing activated B-lymphocytes include a) contacting B-lymphocytes with antigen to obtain immunized cells; b) optionally selecting the immunized cells based on expression of a cell surface marker; c) further contacting the immunized cells with antigen to obtain activated B-lymphocytes.

In some embodiments of the presently disclosed methods, the activated B-lymphocytes subsequently may be transfected with a vector which transforms the transfected cells. In some embodiments, transfecting the activated B-lymphocytes comprises transducing or infecting the activated B-lymphocytes with a virus vector. The activated B-lymphocytes may be transfected and subsequently grown to obtain antibodies that specifically bind to the antigen. Preferably, the activated B-lymphocytes are transfected by infecting the activated B-lymphocytes with a viral vector. Suitable virus vectors may include vectors derived from retroviruses (e.g., lentiviruses), herpes viruses (e.g., Epstein Barr virus and herpes simplex virus type 1), adenoviruses, and adeno-associated viruses.

Suitable virus vectors for use in the methods disclosed herein typically are capable of binding and infecting an activated lymphocyte. For example, the virus vector may include a retrovirus vector that comprises an envelope protein or glycoprotein for binding to a receptor present on B-lineage lymphocytes (e.g., mouse or human B lineage lymphocytes). The virus vector may be obtained by transfecting a suitable packaging cell line with a provirus (e.g., a provirus that expresses one or more oncogenes or protooncogenes). The virus vector may be a pseudotyped virus vector. Suitable packaging cell lines may include the murine psi-2 packaging cell line. In some embodiments, the vector expresses one or more oncogenes or proto-oncogenes. Suitable oncogenes and proto-oncogenes may encode a polypeptide having c-myc activity, a polypeptide having v-abl activity, or both. In preferred embodiments, the vector expresses both a polypeptide having c-myc activity and a polypeptide having v-abl activity. In some embodiments, the vector may express the c-myc polypeptide and the mouse c-myc polypeptide, the v-abl polypeptide, variants of the c-myc polypeptide, or variants of the v-abl polypeptide. Variants of the c-myc polypeptide may include polypeptides having at least about 85% sequence or preferably at least about 95% sequence identity to the human or mouse c-myc polypeptide, where the variant has c-myc polypeptide activity. Variants of the v-abl polypeptide may include polypeptides having at least about 85% sequence identity or preferably at least about 95% sequence identity to the v-abl polypeptide, where the variant has v-abl polypeptide activity.

In other embodiments of the presently disclosed methods, the activated B-lymphocytes subsequently may be fused with a transformed cell (e.g., a myeloma cell) to obtain a hybridoma. The transfected cells or hybridomas may be grown in an in vivo system (e.g., in an animal host) or entirely in vitro under conditions described herein for growing and/or selecting antibody-producing cells.

In further embodiments of the presently disclosed methods, the antibody encoding nucleic acid (e.g., the Ig heavy and light chain sequences) may be molecularly cloned from the activated B-lymphocytes. The Ig heavy and light chains sequences then may be transfected into host cells to produce and obtain antibodies that specifically bind to the antigen.

The methods typically include contacting lymphocytes with an antigen to obtain immunized cells and may include contacting immunized cells with antigen to obtain activated cells. The lymphocytes (or immunized cells) may be contacted directly with antigen or indirectly with antigen via antigen-presenting cells. For example, lymphocytes (or immunized cells) may be contacted with antigen by (i) contacting antigen-presenting cells with the antigen; and (ii) contacting the antigen-presenting cells with the lymphocytes (or immunized cells). Antigen-presenting cells may include dendritic cells, macrophage cells, B cells, or a mixture thereof. Preferably, the antigen-presenting cells comprise dendritic cells. Antigen-presenting cells may be obtained from any suitable source. In some embodiments, antigen-presenting cells are obtained from one or more of spleen cells (e.g., splenocytes), peripheral blood leukocytes, bone marrow cells, and cord blood cells. Preferably, the antibody-presenting cells are obtained from peripheral blood leukocytes.

The methods for producing antibodies may include selecting a clonal population of transformed, activated lymphocytes in vivo or ex vivo. For example*, a clonal population may be selected by growing the cells and selecting those cells that produce an antibody that binds to the antigen specifically. In some embodiments, a clonal population may be selected by transferring and growing the transformed lymphocytes in a host animal (e.g., a SCID mouse). In another example, a clonal population may be selected by culturing the cells in vitro using conditions described herein for growing and/or selecting antibody-producing cells. In some embodiments, the clonal population may be further grown after selection, either in vivo or ex vivo, to obtain antibodies that specifically bind to the antigen.

The methods for producing antibodies may be used to obtain antibodies having a preferred affinity for the antigen. For example, the methods may be used to produce antibodies that specifically bind to the antigen with an affinity (K_{D}) of at least about 10⁶ M⁻¹, preferably at least about 10⁸ M⁻¹, and more preferably at least about 10¹⁰ M⁻¹.

The cells used in the methods disclosed herein may be human or non-human animal cells (e.g., macaque, mouse, rat, or rabbit cells). For example, B-lymphocytes, antigen presenting cells, immunized cells, infected cells, and other cells such as thymoma cells, macrophages, T cells, and stromal cells, may be human or non-human animal cells (e.g., macaque, mouse, rat, and rabbit cells).

Also disclosed are antibodies produced by the aforementioned methods. The antibodies may include monoclonal antibodies that specifically bind to a selected antigen. The antibodies may be formulated as a pharmaceutical composition for treating or diagnosing a disease or condition associated with the antigen. In some embodiments, the antibodies are conjugated to a therapeutic or diagnostic agent. The antibodies disclosed herein may be human antibodies or non-human antibodies (e.g., mouse, rat, and rabbit antibodies).

### BRIEF DESCRIPTION OF THE DRAWINGS

FIG. 1 illustrates embodiments of methods for producing monoclonal antibodies that includes in vivo plasmacytoma development.

FIG. 2 illustrates one embodiment of a method for producing monoclonal antibodies that includes in vitro plasmacytoma development.

FIG. 3 illustrates one embodiment of a method for producing monoclonal antibodies that includes in vitro stimulation of a lymphocyte population and in vivo plasmacytoma development or molecular cloning and expression.

FIG. 4 demonstrates that enrichment of GL7+ cells from murine splenocytes enriches for antigen specific antibody secreting cells as determined by ELISpot, which were performed on serial dilutions of the indicated cells. The number of antigen-specific Ig specific cells per 10⁶ total cells is indicated.

FIG. 5 indicates that GL7-enriched splenocytes can be activated in vitro to produce activated B-lymphocytes in response to antigen. Splenocytes isolated from mice immunized with the α-subunit of E. coli RNA polymerase (Ag) were magnetically selected using an anti-GL7 antibody. Cells were stained with fluorophore-conjugated antibodies against the cell-determinant surface antigens CD45 (lymphocytes), B220 (B cells), CD138 (plasmablasts), CD11b (monocytes/macrophages) and CD3e (T cells) before analysis on a Beckman LSRII benchtop cytometer. Results for B220-positive and CD138-positive cells are shown. Results for CD45-negative, CD3ε-positive and CD11b-positive cells are omitted. CD138-positive cells (activated B-lymphocytes, including plasmablasts) are bracketed and the percentage of CD138-positive cells (as a function of the entire cell population) is indicated.

FIG. 6 illustrates the complex culture requirements for in vitro B cell culture. Conditioned medias were obtained by harvesting the supernatants of cell lines after 12-24 h of stimulation with PMA and PHA. The in vitro B cell cultures were maintained for 8 days with different conditioned medias A. Total cell number (in millions). B. % viability (by trypan). C. Antigen specific spots as determined by ELISpot (per million cells). D. Antigen specific spots as determined by ELISpot for different concentrations of EL4-B5 conditioned media. Optimizing antigen dosage for in vitro culture. In vitro cultures were maintained for up to 12 days to assess the effects of different concentrations of antigen. E. Viability as determined by trypan blue exclusion. F. Antigen specific spot frequency as determined by ELISpot. G. Ratio of antigen specific spots versus total IgG spots as determined by ELISpot.

FIG. 7 demonstrates ABL-MYC transformation of activated B-lymphocytes in vitro. Splenocytes were treated with anti-CD40 and interleukins 4, 5 and 6 before injection with ABL-MYC. Infected cells were plated on OP-9 feeder cells in the presence of 10 ng/ml Il-6. A. After 21 (C1) and 45 (C2) days, two cell lines were isolated. Cells shown were gated for single cells. No CD3- or F4/80 CD11b-positive cells were observed. B. Histogram of CD138 expression for the samples shown in A. Control indicates non-stained C2 cells. C. Cell lines C1 and C2 contain integrated ALB-MYC. Genomic DNA was prepared from cell extracts and subjected to PCR analysis for ABL-MYC integration. A plasma containing ABL-MYC and plasmacytoma line 177E are shown as positive controls. The negative (−) control contained no DNA in the reaction tube. D. Cell lines C1 and C2 secrete IgG. Supernatants from 4- to 7-day cultures were analyzed for total IgG.

FIG. 8 demonstrates murine splenocyte upregulation of CD138 in response to anti-CD40 and interleukins 4, 5, and 6. Splenocytes were cultured in the presence of 1 μg/mL anti-CD40, 150 ng/mL IL-4, 10 ng/mL IL-15, and 10 ng/mL IL-6. After 4 days, cells were collected and stained with APC-Cy7-labeled anti-CD45, PE-Cy5.5-labeled anti-CD19, PE-labeled anti-CD138, FITC-labeled anti-CD3, and APC-labeled anti-F4/80 and anti-CD11b before analysis by flow cytometry. A. B220 and CD138 expression profiles of freshly isolated splenocytes. B. B220 and CD138 expression profiles of splenocytes depleted of CD138, then after 2 and 4-days of treatment as indicated. Cells shown were gated for single cells and CD45 (lymphocytes)-positive cells. Cells expressing CD3 (T-cells) and F4/80/CD11b (macrophages) were excluded from the analysis

FIG. 9 demonstrates GL7 selection on human lymphocytes propagated in a NOD-SCID mouse. A flow cytometry analysis of whole splenocytes, the GL7 depleted population and the GL7 selected population. The first row depicts the FSC vs. SSC profile. The second row depicts histograms of the cells gated in the first row for human CD45 expression. The last row depicts huCD45+ population illustrating expression of the B cell marker CD19 and the activation marker CD38. B. Table with total cell numbers for each of the populations and IgG or antigen specific antibody secreting cells, as determined by ELISpot, and the estimate of the antibody secreting cells in the entire population. C. Representative ELISpot of cell populations. The first 2 columns show antigen specific spots. The middle 2 columns are a non-specific control protein, and the final two columns show total IgG secreting spots.

FIG. 10. Demonstrates that human cells generated as described in FIG. 9, can be sorted, and are amenable to single cell RT-PCR to generate antibodies. A. Flow cytometric plot of CD19+ antigen binding cells. B. The products of scRT-PCR (from single cells collected by flow cytometry) run on a gel. Lanes 4 and 5 demonstrate CDR regions of a heavy and a light chain from a single cell (labeled Cell 13 Heavy and Cell 13 Light).

FIG. 11. illustrates bio-layer interferometry analysis to measure the nanometer shift in light wavelength due to changes in thickness at the probe surface caused by an antibody binding to a ligand. A. Bio-layer interferometry data from the analysis of negative controls. B. Biolayer interferometry data from the analysis of samples shows association of anti-human antibody to the antibodies generated using the CDR analyzed in FIG. 10.

### DETAILED DESCRIPTION

The subject matter disclosed herein is described using several definitions, as set forth below and throughout the application.

Unless otherwise noted, the terms used herein are to be understood according to conventional usage by those of ordinary skill in the relevant art. In addition to the definitions of terms provided below, it is to be understood that as used in the specification, embodiments, and in the claims, “a”, “an”, or “the” can mean one or more, depending upon the context in which it is used.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≦10% of the particular term and “substantially” and “significantly” will mean plus or minus>10% of the particular term.

As used herein, the terms “include” and “including” have the same meaning as the terms “comprise” and “comprising.” These terms should be interpreted to be “open-ended” unless otherwise specified.

As used herein, the term “antibody” refers to a protein comprising at least one, and preferably two, heavy (H) chain variable regions (abbreviated as VH), and at least one and preferably two light (L) chain variable regions (abbreviated as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined. (See, e.g., Kabat, E. A., e.g. (1991) Sequences of Proteins of Immunological Interest, Fifth Edition, U.S. Department of Health and Human Services, NIH Publication No. 91 3242; and Chothia, C. e.g. (1987) J. Mol. Biol. 196:901-917; which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The term “antigen-binding fragment” of an antibody (or simply “antibody portion” or “fragment”), as used herein, refers to one or more fragments of a full-length antibody that retain the ability to specifically bind to the antigen. Examples of antigen-binding fragments of the disclosed antibodies include, but are not limited to: (i) an Fab fragment or a monovalent fragment consisting of the VL, VH, CL and CH1 domains; (ii) an F(ab′)₂ fragment or a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment (Ward e.g., (1989) Nature 341:544 546), which consists of a VH domain; and (vi) an isolated complementarity determining region (CDR). Even though the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or “scFv.” (See, e.g., Bird e.g. (1988) Science 242:423 426; and Huston e.g. (1988) Proc. Natl. Acad. Sci. USA 85:5879 5883). Single chain Fv or “scFv” are encompassed within the term “antigen-binding fragment” of an antibody.

The disclosed antibodies can be full-length (e.g., an IgG (e.g., an IgG1, IgG2, IgG3, IgG4), IgM, IgA (e.g., IgA1, IgA2), IgD, and IgE) or can include only an antigen-binding fragment (e.g., a Fab, F(ab′)₂ or scFV fragment, or one or more CDRs). The antibodies disclosed herein may be a polyclonal or monoclonal antibodies. The disclosed antibodies may be monospecific, (e.g., a monoclonal antibody, or an antigen-binding fragment thereof), or may be multispecific (e.g., bispecific recombinant diabodies). In some embodiments, the antibody can be recombinantly produced (e.g., produced by phage display or by combinatorial methods). In some embodiments, the antibodies (or fragments thereof) are recombinant or modified antibodies (e.g., a chimeric, a humanized, a deimmunized, or an in vitro generated antibody).

The terms “monoclonal antibody” or “monoclonal antibody composition” as used herein refer to a preparation of antibody molecules of single molecular composition. A monoclonal antibody composition displays a single binding specificity and affinity for a particular epitope. Accordingly, the term “monoclonal antibody” refers to antibodies displaying a single binding specificity which have variable and constant regions derived from germline immunoglobulin sequences.

The terms “antibody-producing cell” and “plasma cell” may be used interchangeably herein and refer to a type of white blood cell of the B-cell lineage that produces and secretes antibodies. As used herein “antibody-producing cell” and “plasma cell” may include immortalized antibody-producing cells and immortalized plasma cells. An antibody-producing cell or a plasma cell may be “antigen-specific” whereby the cell produces antibodies that bind specifically to a given antigen.

As used herein, “lymphocytes” are defined as cells involved in vertebrate immunity. These cells include the B lymphocytes (B cells), T lymphocytes (T cells), dendritic cells, and natural killer cells. Lymphocytes include immune cells isolated from blood, spleen, and lymph nodes. B-lymphocytes are immunoglobulin-expressing lymphocytes. These cells include, but are not limited to naïve B cells, memory B cells, plasmablasts, and plasma cells. The B-lymphocytes may actively secrete immunoglobulin or display the immunoglobulin on their cell surface. B-lymphocytes can express immunoglobulin M (IgM), immunoglobulin G (IgG), immunoglobulin A (IgA), immunoglobulin E (IgE), and immunoglobulin D (IgD). Activated, antigen-specific B-lymphocytes are said to express immunoglobulin with a high affinity for antigen. Activated lymphocytes are defined as lymphocytes which are actively undergoing cell division and expansion. Activated lymphocytes recognize antigen, present antigen, or express immunoglobulin against antigen in response to activating agents. Activating agents include, but are not limited to, antigen, cytokines, immunoglobulin, and/or other cells (e.g., lymphocytes).

As used herein, “specific binding” refers to antibody binding to a predetermined antigen. Typically, the antibody binds with an affinity corresponding to a K_{D} of about 1×10⁻⁶ M or less, and binds to the predetermined antigen with an affinity corresponding to a K_{D} that is at least two orders of magnitude lower than its affinity for binding to a non-specific antigen (e.g., BSA, casein) other than the predetermined antigen or a closely-related antigen. The term “K_{D}” (M), as used herein, is intended to refer to the dissociation equilibrium constant of a particular antibody-antigen interaction.

“Percentage sequence identity” may be determined by aligning two sequences of equivalent length using the Basic Local Alignment Search Tool (BLAST) available at the National Center for Biotechnology Information (NCBI) website (i.e., “bl2seq” as described in Tatiana A. Tatusova, Thomas L. Madden (1999), “Blast 2 sequences—a new tool for comparing protein and nucleotide sequences”, FEMS Microbiol Lett. 174:247-250, incorporated herein by reference in its entirety). For example, percentage sequence identity between two sequences may be determined by aligning the sequences using the online BLAST software provided at the NCBI website. “Percentage sequence identity” between two deoxyribonucleotide sequences may also be determined using the Kimura 2-parameter distance model which corrects for multiple hits, taking into account transitional and transversional substitution rates, while assuming that the four nucleotide frequencies are the same and that rates of substitution do not vary among sites (Nei and Kumar, 2000) as implemented in the MEGA 4 (Tamura K, Dudley J, Nei M & Kumar S (2007) MEGA4: Molecular Evolutionary Genetics Analysis (MEGA) software version 4.0. Molecular Biology and Evolution 24:1596-1599), preferably version 4.0.2 or later.

As used herein, “transfecting” means introducing nucleic acid into a cell. “Transfecting” may include “infecting” and “transducing.”

As used herein, “immortalization” means conversion of a normal cell into a cancerous cell. Immortalization may be performed by fusing a normal cell to a cancerous cell to obtain an immortalized hybrid cell or hybridoma. Immortalization also may be performed by “transformation”, which means genetic alteration of a normal cell to a cancerous cell resulting from the introduction, uptake and expression of foreign genetic material. Immortalization may be performed by infecting a normal cell with a transforming viral vector to obtain a plasmacytoma.

As used herein, “ABL-MYC virus” refers to a replication defective retrovirus which contains v-abl form the Abelson Murine Leukemia virus (Ab-MuLV) and murine c-myc. (See U.S. Pat. Nos. 5,244,656 and 5,705,150, which are incorporated by reference herein in their entireties.) As used herein, “c-myc activity” and “v-abl activity” may include transforming activity.

As used herein, “molecular cloning” includes obtaining antibody-encoding sequences used for generating antibodies by reverse transcribing Ig heavy and light chain RNA from antibody-producing cells into cDNA, and then amplifying the cDNA by polymerase chain reaction. The heavy and light chain cDNAs thus obtained may then be cloned into expression vectors which may be utilized to produce full antibodies via transient and stable transfections of host cells.

Lymphocytes and Antigen-Presenting Cells

Lymphocytes (e.g., B-lymphocytes) and antigen-presenting cells may be obtained from any suitable source including non-human animals or human animals. For example, lymphocytes and antigen-presenting cell may be present within spleen cells (e.g., splenocytes), blood peripheral leukocytes, cord blood cells, and bone marrow cells. Lymphocytes may include B-lineage lymphocytes and T-lymphocytes. Antigen-presenting cells typically include dendritic cells, macrophage cells, and B-lineage lymphocytes.

In some examples, lymphocytes may be obtained from transgenic animals. For example, a lymphocyte may include a B cell obtained from a transgenic or transchromosomal non-human animal, e.g., a transgenic mouse, having a genome comprising a human heavy chain transgene and a light chain transgene.

Lymphocyte populations may be contacted with antigens such as recombinant proteins and peptides of interest. Antigen contact may occur in vitro or in vivo, and may include special adjuvants and/or dendritic cells. Antigen contact may result in activation or selection of antigen specific cell populations.

Lymphocytes and antigen-presenting cells may be selected using fluorescence-activated cell sorting (FACS) based on selected cell markers. For example, a cell population may be enriched in or depleted of a subpopulation that includes a selected marker for example, the antigen detected by the antibody GL7 (i.e., Neu5Ac α2-6 LacNAc-containing N-glycan), CD38, CD20, CD138, CD40, CD45, CD3 (e.g., CD3e), CD11 (e.g., CD11b or CD11c), CD19, F4/80, CD79, B220, CD14, and CD86.

Virus Vectors

The term “vector,” as used herein, is intended to refer to a nucleic acid molecule capable of transporting another nucleic acid to which it has been linked, i.e., a “transgene.” One type of vector is a “viral vector,” wherein additional nucleic acid segments or transgenes may be ligated into the viral genome or a portion of the viral genome and packaged into a virus or a replication defective virus. Viral vectors may include replication defective retroviruses, adenoviruses, adeno-associated viruses, and herpesviruses. A viral vector typically is capable of binding and entering a host cell and thereafter expressing a transgene. As such, the genome of a viral vector typically includes the minimum cis-elements for packaging the genome and expressing any transgene present therein. A viral vector may be prepared by transfecting a packaging cell line with a plasmid that includes the minimum cis-elements for packaging the genome and expressing any transgene present therein. The packaging cell line typically produces all of the proteins necessary for producing the virus vector (i.e., the trans-factors). The virus vector produced by a packaging cell line may include a homologous envelope glycoprotein or a heterologous envelope glycoprotein (i.e., the virus vector may be “pseudotyped”), which may alter the host cell range for the viral vector relative to the virus from which the virus vector is derived.

Retrovirus vectors are derived from retroviruses. The retrovirus genome and the associated proviral DNA minimally have three genes: gag, pal and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTR's serve to promote transcription and polyadenylation of the virion RNA's. The LTR typically contains all other cis-acting sequences necessary for viral replication. Some retroviruses, such as lentiviruses, have additional genes includine vif, vpr, tat, rev, vpu, lief and vpx (in HIV-1, HIV-2 and/or SIV).

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

A packaging cell may be prepared by transfecting a suitable host cell with a first vector encoding a viral gag and a viral pal and another vector encoding a viral env. A packaging cell line may be prepared by transfecting a vector containing viral gag, pal, and env on a single vector. Suitable packaging cells include murine psi-2 cells. The viral env may be homologous or heterologous. Introducing a nucleic acid that includes the cis-elements and optionally a transgene, herein referred to as a “transfer vector,” into the packaging cell yields a producer cell, which releases infectious virus vector particles carrying the foreign gene of interest. The transfer vector may be transiently transfected or stably transfected into the packaging cell line.

Virus vectors are known in the art, see, e.g., Weissinger, et al., PNAS (1991), 88:8735-8739; Naldini et al., Sci. (1996) 272:263 267; and Zufferey et al., Nat. Biotech. (1997) 15:871 875. Generally the vectors are plasmid-based or virus-based, and are configured to carry the essential sequences for incorporating foreign nucleic acid, for selection and for transfer of the nucleic acid into a host cell. The gag, pol and env genes of suitable vectors also are known in the art. Thus, the relevant genes may be cloned into a selected vector and then used to transform the target cell of interest.

The env gene can be derived from any virus, including retroviruses. Examples of retroviral-derived env genes include, but are not limited to: gibbon ape leukemia virus (GaLV or GALV); Moloney murine leukemia virus (MoMuLV or MMLV), Harvey murine sarcoma virus (HaMuSV or HSV), murine mammary tumor virus (MuMTV or MMTV), human immunodeficiency virus (HIV) and Rous sarcoma virus (RSV). The env gene may encode an amphotropic envelope protein, which allows transduction of cells of human and other species. Other env genes' such as Vesicular stomatitis virus (VSV) protein G (VSV G) or that of hepatitis viruses and of influenza also may be used.

It may be desirable to target the virus vector by linking the envelope protein with an antibody or a particular ligand for targeting the virus vector to antibody-producing cells (e.g., activated B-lymphocytes). Targeting may be accomplished by using an antigen-binding portion of an antibody or a recombinant antibody-type molecule, such as a single chain antibody, to target the retroviral vector to an antibody-producing cell.

The transgene can be any nucleic acid of interest which can be transcribed. Generally the transgene encodes a polypeptide. Preferably, expression of the polypeptide has some desirable effect. Transgenes of the virus vectors disclosed herein may include oncogenes and proto-oncogenes for transforming antibody-producing cells.

A transgene may be expressed from a promoter sequence present in the LTR region of the vines from which the virus vector is derived. Alternatively, the transgene may be expressed from another promoter sequence. The promoter sequence may be homologous or heterologous to the transgene sequence. A wide range of promoters may be utilized, including a viral or a mammalian promoter. Cell or tissue specific promoters can be utilized to target expression of gene sequences in specific cell populations. Preferably, the selected promoter expresses the transgene in antibody-producing cells.

Optionally, the nucleic acid of the virus vector contains a marker gene. Marker genes may be utilized to assay for the presence of the vector, and thus, to confirm infection and integration. The presence of a marker gene ensures the selection and growth of only those host cells which express the inserts. Genes for selecting or sorting may encode proteins that confer resistance to antibiotics and other toxic substances, e.g., histidinol, puromycin, hygromycin, neomycin, methotrexate, and cell surface or other markers (e.g., green fluorescent protein (GFP)).

The recombinant virus of the invention is capable of transferring a nucleic acid sequence into a mammalian cell. The term, “nucleic acid sequence”, refers to any nucleic acid molecule, preferably DNA, as discussed in detail herein. The nucleic acid molecule may be derived from a variety of sources, including DNA, cDNA, synthetic DNA, RNA or combinations thereof. Such nucleic acid sequences may comprise genomic DNA which may or may not include naturally occurring introns. Moreover, such genomic DNA may be obtained in association with promoter regions, poly A sequences or other associated sequences. Genomic DNA may be extracted and purified from suitable cells. Alternatively, messenger RNA (mRNA) can be isolated from cells and used to produce cDNA by reverse transcription or other means.

The virus vectors are prepared by introducing the vector nucleic acid via transfection or infection into the packaging cell line. The packaging cell line produces vector viral particles that contain the vector nucleic acid (i.e., the transfer vector). The virus vector may be recovered from the culture media of the packaging cell line and titered by standard methods.

Stable cell lines where the packaging functions are configured to be expressed by a suitable packaging cell are known. For example, see Mann et al., Cell (1983), 133:153-165; and Ory et al., Proc. Natl. Acad. Sci. (1996) 93:11400 11406, which describe packaging cells.

Oncogenes and Proto-Oncogenes

The disclosed methods may include transfecting (e.g., by transducing or infecting) antibody-producing cells with one or more vectors (e.g., a virus vector) that express one or more oncogenes or proto-oncogenes. Subsequently, the antibody-producing cells may become transformed. In some embodiments of the disclosed methods, antigen-specific plasmacytomas develop subsequent to the expression of one or more oncogenes or proto-oncogenes in the transfected antibody-producing cells. Suitable oncogenes or proto-oncogenes for the present methods include those encoding c-myc, v-abl, Rb, p53, h-tert, v-fms, c-mos, mdm-2, p16/p19, v-ras, v-raf, and members of the bcl-2 family (e.g., bcl-xl). The preferred oncogenes or proto-oncogenes are those encoding c-myc, mouse c-myc, and v-abl. The oncogenes or proto-oncogenes may be expressed via an endogenous promoter for the oncogene or a heterologous promoter that effects expression of the oncogene or proto-oncogene in antibody-producing cells.

Screening and Selection for Immortalized Plasma Cells

After transfection (or transduction or infection), immortalized or transformed cells may be selected in vivo or ex vivo. In order to select for transformed cells in vitro (or ex vivo), the transfected cells may be grown and/or selected under conditions described herein for growing and/or selecting immunized cells. To select for transformed antibody cells ex vivo, the cells may be plated at approximately 1×10⁵ per well in flat bottom microtiter plate. Individual wells can then be screened by ELISA for kappa-light chain containing antibodies, by Octet for specificity and affinity, and by FACS analysis to identify cells that produce antibody to the selected antigen. Antibody-secreting cells can be re-plated, screened again, and if still positive for IgG, the cells can be grown further ex vivo or in vivo to generate antibody for characterization.

In order to select for transformed cells in vivo, the cells may be transferred to the peritoneal cavity of a mouse. Prior to transfer, the mouse optionally may have been primed with a suitable agent for inducing or enhancing production of ascitic fluid. After the cells are transferred, the mouse is monitored for production of ascitic fluid, which may be screened for the presence of antibody.

The presence of the antibodies produced by the methods disclosed herein may be determined by various assays. Assay techniques include but are not limited to immunofluorescence (IF) by cytofluorographic analysis or by cell sorting, indirect immunofluoroscence, immunoprecipitation, ELISA, agglutination, affinity and Western blot techniques.

### ILLUSTRATIVE EMBODIMENTS

The following embodiments are illustrative and are not intended to limit the scope of the disclosed subject matter.

### Embodiment 1

A method for producing antigen-specific plasma cells, comprising: a) immunizing an animal and isolating lymphocytes from the animal; b) isolating lymphocytes positive for the cell surface antigen recognized by an anti-GL7 antibody to obtain immunized cells; c) contacting the immunized cells with an activating agent to obtain activated, antigen-specific B-lymphocytes.

### Embodiment 2

The method of embodiment 1, wherein the animal is a mouse.

### Embodiment 3

The method of embodiment 1 or 2, wherein the activating agent comprises the antigen.

### Embodiment 4

The method of embodiment 3, wherein the activating agent further comprises antigen-specific T-cells.

### Embodiment 5

The method of any of the foregoing embodiments, wherein the activating agent further comprises dendritic cells.

### Embodiment 6

The method of any of the foregoing embodiments, wherein the activating agent further comprises macrophages.

### Embodiment 7

The method of any of the foregoing embodiments, wherein the activating agent further comprises an antibody.

### Embodiment 8

The method of embodiment 7, wherein the antibody is an antibody against CD40.

### Embodiment 9

The method of any of the foregoing embodiments, wherein the immunized cells and the activating agent are contacted for at least about 2 days.

### Embodiment 10

A method for producing antigen-specific plasma cells, comprising: a) engrafting an animal with a population of cells; b) immunizing an animal and isolating lymphocytes from the animal; c) isolating lymphocytes positive for the cell surface antigen recognized by an anti-GL7 antibody to obtain immunized cells; d) contacting the immunized cells with an activating agent to obtain activated, antigen-specific B-lymphocytes and the separation of B-lymphocytes into individual cells.

### Embodiment 11

The method of embodiment 10, further comprising separating the activated, antigen-specific B-lymphocytes into single cells.

### Embodiment 12

The method of embodiment 10 or 11, wherein the activating agent further comprises antigen-specific T-cells, dendritic cells, macrophages, or a mixture thereof.

### Embodiment 13

The method of any of embodiments 10-12, wherein the activating agent further comprises a cytokine.

### Embodiment 14

The method of embodiment 13, wherein the cytokine is IL-21.

### Embodiment 15

The method of any of the embodiments 10-14, wherein the activating agent further comprises an antibody.

### Embodiment 16

The method of embodiment 15, wherein the antibody is an antibody against CD40.

### Embodiment 17

The method of any of the embodiments 10-16, wherein the activating agent comprises the TLR-9 agonist.

### Embodiment 18

The method of embodiment 17, wherein the TLR-9 agonist is ODN2006.

### Embodiment 19

The method of any of the embodiments 10-18, wherein step a) comprises engrafting an animal with cells.

### Embodiment 20

The method of embodiment 19, wherein the engrafted cells are human lymphocytes.

### Embodiment 21

The method of any of the embodiments 10-20, wherein the engrafted human lymphocytes are depleted for CD8.

### Embodiment 22

The method of any of the embodiments 10-21, wherein the animal is a mouse.

### Embodiment 23

The method of embodiment 22, wherein the animal is a mouse, which lacks a functional immune system.

### Embodiment 24

The method of embodiment 23, wherein the animal is a SCUD-NOD mouse.

### Embodiment 25

The method of any of the embodiments 10-24, wherein step d) comprises separating the activated, antigen-specific B-lymphocytes into single cells.

### Embodiment 26

The method of embodiment 25 wherein each single activated, antigen-specific B-lymphocyte can be processed into nucleic acid molecules representing the binding domain of the antigen-specific B-lymphocytes.

### Embodiment 27

The method of embodiment 26 wherein the binding domain represents both the heavy and light chain of each single activated, antigen-specific B-lymphocyte can be PCR amplified.

### Embodiment 28

The method of embodiment 27, wherein the heavy chain variable and constant regions, and the light chain variable and constant regions are molecularly cloned.

### Embodiment 29

The method of embodiment 28, further comprising expressing the antibody in a cell transfected with the molecularly cloned heavy chain variable and constant regions, and the light chain variable and constant regions.

### Embodiment 30

The method of embodiment 29, further comprising isolating the antibody.

### EXAMPLES

The following examples are illustrative and are not intended to limit the disclosed subject matter.

### Example 1

### Method for Plasmacytoma Development In Vivo or In Vitro

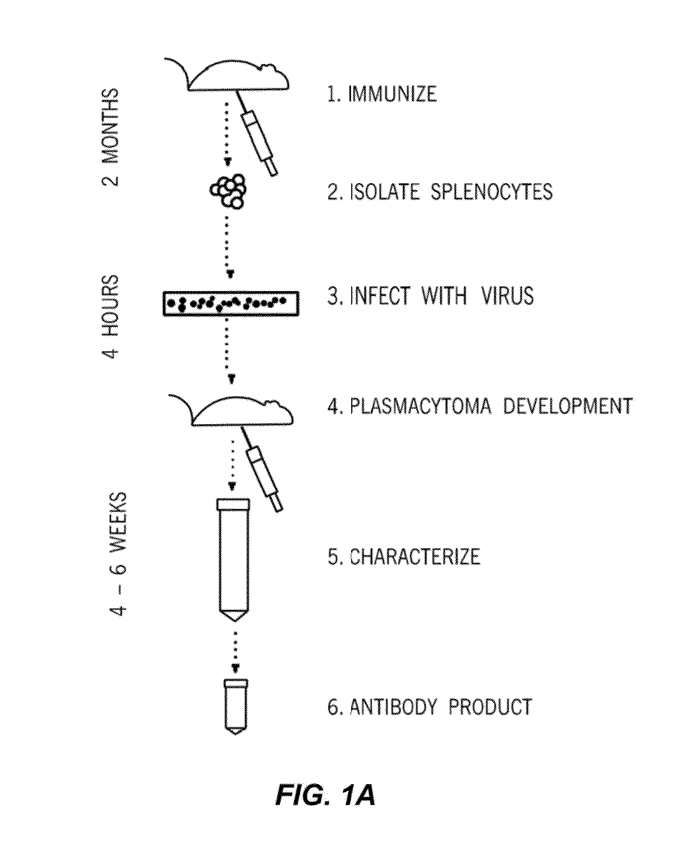

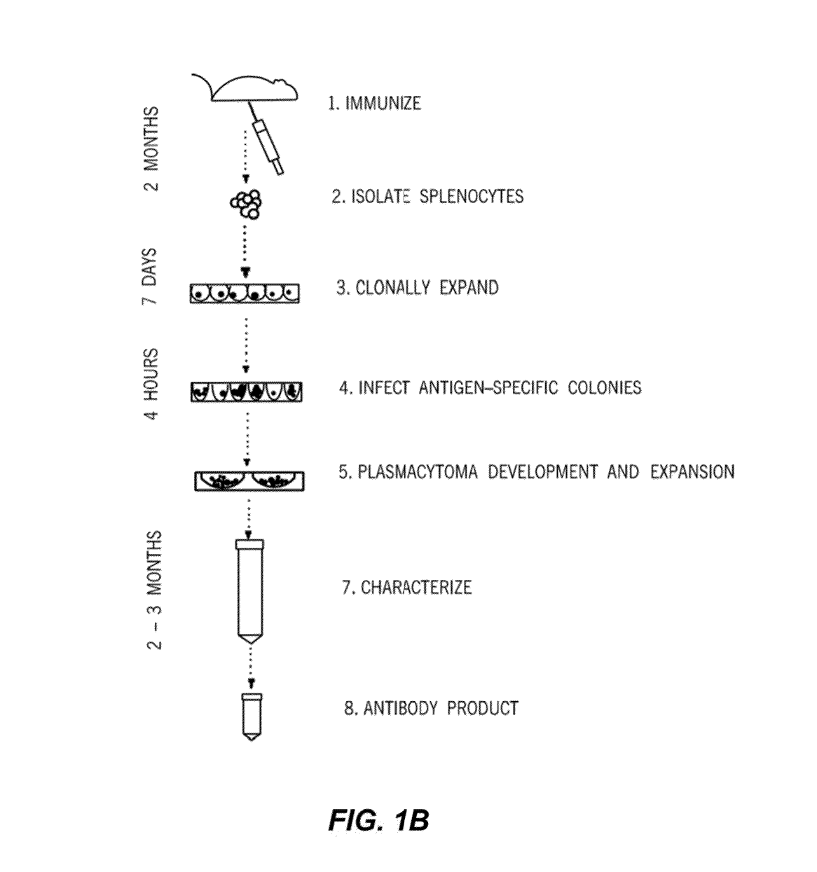

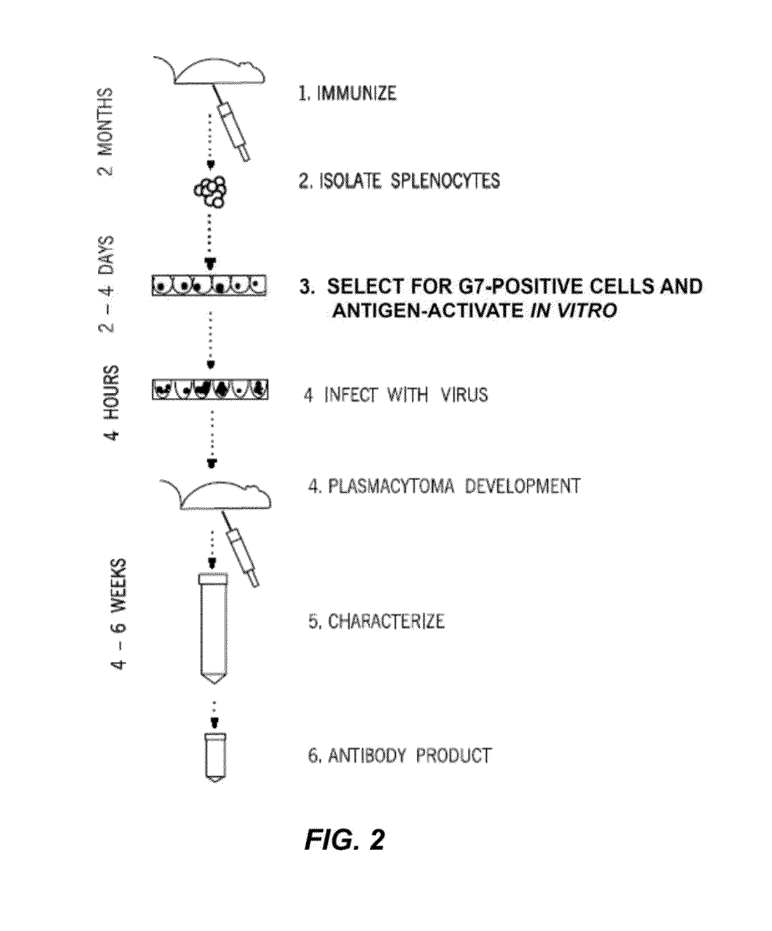

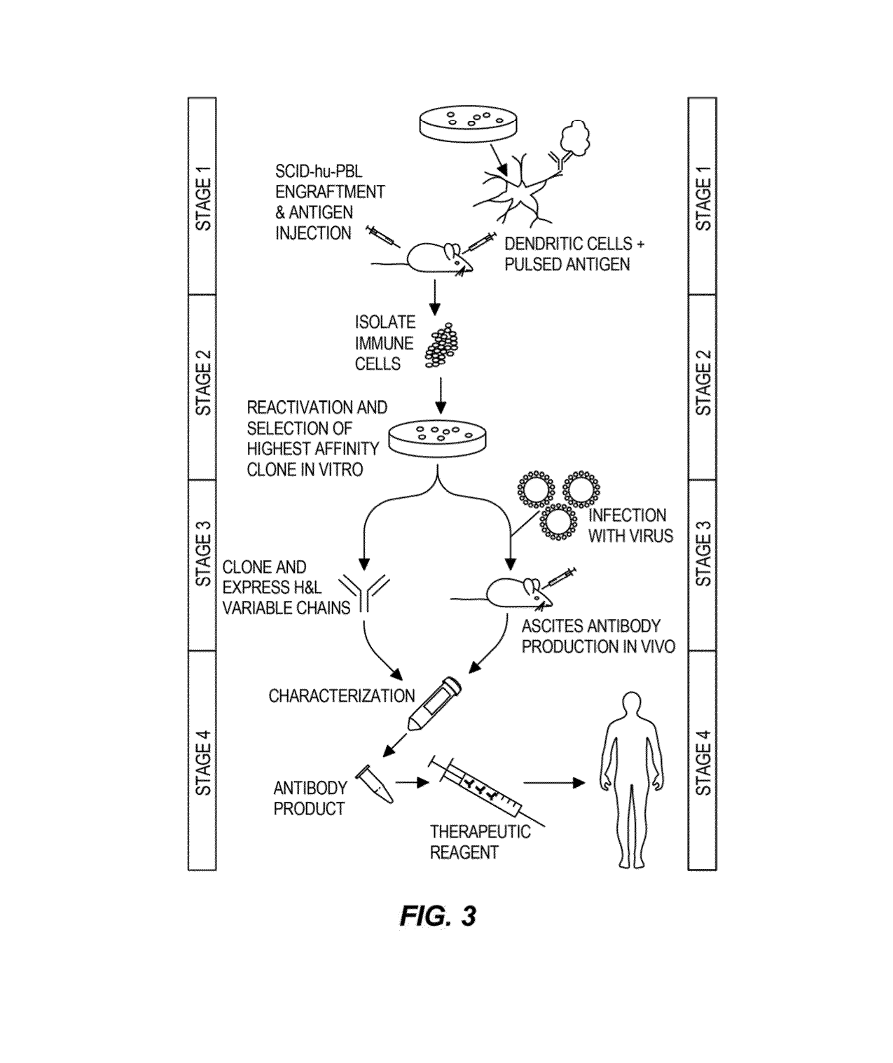

FIGS. 1-3 outline exemplary methods for producing monoclonal antibodies. In the method of FIG. 3, B cells from immunized mice are clonally expanded for several days to induce differentiation and Ig secretion. Colonies that secrete Ig against the immunizing antigen are infected with a ABL-MYC retrovirus and then culture in vitro for plasmacytoma development and expansion

### Example 2

### Transformation of GL7-Enriched Cells by ABL-MYC Retrovirus

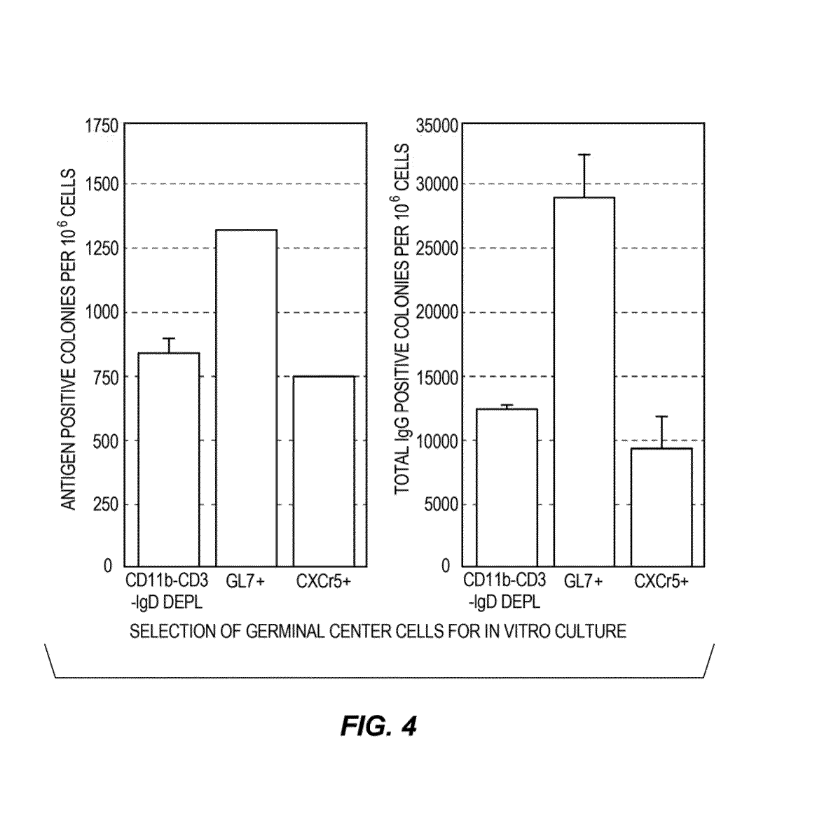

Splenocytes isolated from mice immunized with the α-subunit of E. coli RNA polymerase were fractionated with the anti-GL7 antibody conjugated to fluorescein isothiocyanate (FITC) and anti-FITC magnetic beads (FIG. 4). Ag-specific ELISPOT data indicated that Ag-specific, IgG secreting cells were enriched more than ten-fold in the GL7-enriched population when compared to non-fractionated splenocytes (total splenocytes). These data indicate that the magnetically labeled cells were enriched for antigen specific cells responding to immunogen boost.

Total splenocytes, GL7-enriched, and GL7-depleted populations were infected with ABL-MYC retrovirus and injected into recipient mice. (Table 1)

TABLE 1
Characterization of ascites development from ABL-MYC-
infected splenic and in vitro cell cultures.

| | Ag- | | | | | ABL- | IgG- |
|---|---|---|---|---|---|---|---|
| | specific | | | | Ag- | MYC- | positive |
| | IgG | Cells | | Ascites | specific | positive | ascites |
| | secretion | injected/ | Avg. | developed | ascites | ascites | (% of |
| | (spots/10⁶ | mouse | latency | (% of total | (% of total | (% of total | total |
| Condition | cells) | (×10⁶) | (days) | recipients) | ascites) | ascites) | ascites) |
| Total | 750 | 2.5 | 50 | 100 | 83 | 100 | 100 |
| splenocytes | | | | | | | |
| GL7⁺ | 1200 | 2.5 | 34 | 100 | 100 | 100 | 100 |
| GL7⁺ day 4 | 1000 | 0.5 | 72 | 100 | 100 | 100 | 100 |
| GL7⁺ day 8 | 2200 | 1.5 | 87 | 67 | 100 | 100 | 100 |

In Table 1, the indicated cell populations were infected with ABL-MYC, washed, and injected into six recipient mice along with non-infected splenocyte carriers isolated from Lysozyme-immunized mice. Ascites were collected from all mice, except 2 mice in the 8-day cultures, which did not develop ascites within 90 days and were sacrificed. Latency is the time required for ascites development from the time of injection. Ag-specific titer was calculated by α-core-specific ELISA and is defined as the dilution that gives half-maximal absorbance. A negative equals an absorbance less than twice background. ABL-MYC integration was carried out as described herein. A positive indicates a clearly visible PCR band upon electrophoretic separation and staining.

Table 1 shows that all of the recipient mice injected with GL7 enriched cells that developed ascites contained Ag-specific IgG. Furthermore, the cells contained within the ascites were found to have the ABL-MYC provirus integrated into the cellular DNA.

### Example 3

### Splenocytes Up-Regulate CD138 in Response to Anti-CD40 and Cytokines

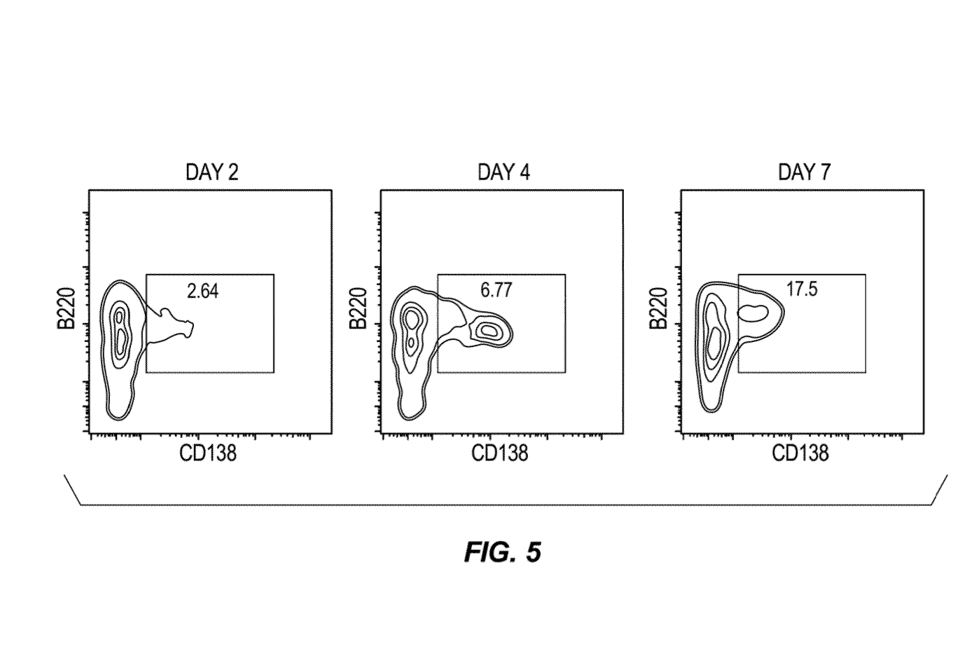

Splenocytes were harvested from immunized mice and cultured with a mAb against CD40 and IL-4, IL-5, and IL-6 for 7 days. Cells were then collected and stained for flow cytometry analysis with antibodies against CD45 (panlymphocyte marker), CD3 (T cell marker), F4/80 and CD11b (macrophage/monocyte markers), CD19 (B cell lineage marker), and CD138 (plasma cell marker) on days 2, 4 and 7 (FIG. 5). These results indicate that in vitro culture systems may be used to activate and differentiate splenocytes into the cell types needed for plasmacytoma development.

### Example 4

### Isolation of ABL-MYC Transformed Plasmacytomas Upon Infection of In Vitro Differentiated Splenocytes

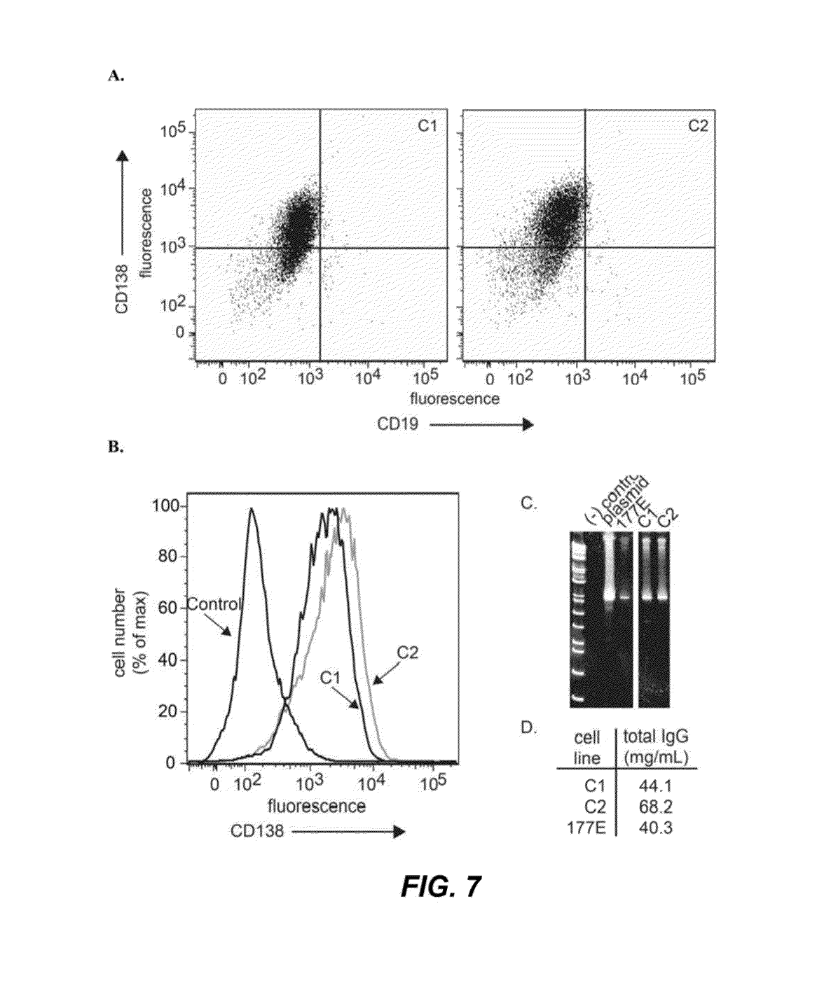

Splenocytes were cultured as described in Example 3 and FIG. 7 and infected with ABL-MYC before plating onto γ-irradiated OP-9 feeders in the presence of 10 ng/mL interleukin (IL)-6. Two cell lines were isolated from primary cultures at days 21 (designated C1) and 45 (C2) and further propagated on OP-9 feeders. After two (C2) and three (C1) passages, cells Were collected and stained for flow cytometry as described in Example 3, FIG. 7. Both cell lines were found to exhibit high CD138 expression and low CD19 expression as expected for a plasma cell line (FIGS. 7A and B). In addition, both cell lines had the ABL-MYC viral gene integrated into their genome (FIG. 7C). Both cell lines secreted IgG into the media (FIG. 7D). These results indicate that ABL-MYC dependent, transformed plasmacytoma lines can be isolated from in vitro cultures.

### Example 5

### Cell Determinate Labeling and FACS

Naïve and memory B cells, plasmablasts and plasma cells can be distinguished by the expression of cell surface markers (Table 2). Because CD138^{high} cells may be the target of ABL-MYC transformation (see Table 1), CD138^{low}/B220^{high} naïve and memory cells may be separated from CD138^{high}/B220^{intermediate} plasmablasts and CD138^{high}/B220^{low} plasma cells. Optionally, plasmablasts and plasma cells can be separated based on B220 expression or naïve and memory B cells can be separated based on IgD expression. (Table 2)

TABLE 2
B lineage cell surface determinant markers

| B cell type | Surface markers |
|---|---|
| Naïve | B220^{high}, IgD^{high}, CD138^{low} |
| Memory | B220^{high}, IgD^{low}, CD 138^{low} |
| Plasmablast | B220^{intermediate}, IgD^{low}, CD138^{high} |
| Plasma | B220^{low}, IgD^{low}, CD138^{high} |

Splenocytes isolated from immunized mice can be labeled with fluorophore-conjugated mAbs directed against the cell surface markers (Tables 2 and 3) using standard protocols for cytometry staining. Plasma cells were observed not to express high levels of B220 or CD19.

TABLE 3
Fluorophore-conjugated mAbs for analysis of lymphocytes.

| Marker | Cell identifier | Fluorophore | mAb clone |
|---|---|---|---|
| B220 | B cell lineage | APC-Cy5.5 | RA3-6B2^{b} |
| IgD | Naive B cell | FITC | 11-26^{c} (11-26)^{b} |
| CD138 | Plasmablast, plasma | PE | 281-2^{a} |
| CD19 | B cell lineage | PerCP-Cy5.5 | 6D5^{b} |
| CD3e | T cell | FITC | 145-2C11^{a} |
| CD11b | Monocyte/macrophage | PE-Cy7 | M1/70^{b} |
| CD45 | Leukocyte | APC-Cy7 | 30-F11 |

^{a}BD Biosciences (San Jose, CA); ^{b}eBioscience (San Diego, CA).

Live, labeled cells may be sorted on a BD FACSVantage SE equipped with the FACSDiva digital electronics package. Cells will be sorted into cooled collection tubes containing fetal calf serum (FCS). Confirmation of sorting and assessment of purity may be carried out by running a small fraction of the sorted populations on a BD LSRII bench top cytometer. In addition to cells that express CD138^{high} or B220^{high}, cells that are neither CD138^{high} nor B220^{high} may be collected as well. Sorted populations may be tested for total and Ag-specific Ig secretion by ELISPOT.

### Example 6

### ABL-MYC Infection

Sorted populations are centrifuged, washed, and resuspended to 4×10⁶ cells per mL in infection media (RPMI 1640 supplemented with 100 U/mL penicillin/streptomycin, 2 mM L-glutamine, 50 μMβ-mercaptoethanol and 20% FCS). The cells are combined with an equal volume of ABL-MYC virus and incubated at 37° C. for 4 h in 5% CO₂. Cells will be washed 3 times in phosphate buffered saline (PBS).

### Example 7

### Transplantation and Ascites Development

Cells are injected intraperitoneally into BALB/c female recipient mice. The recipient mice are primed with 0.5 mL of pristane 7-10 days before receiving infected cells. Upon injection, infected cells are incubated for up to 60 days with typical ascites development occurring within 30-45 days. Ascites fluid and cells are collected upon development for analysis.

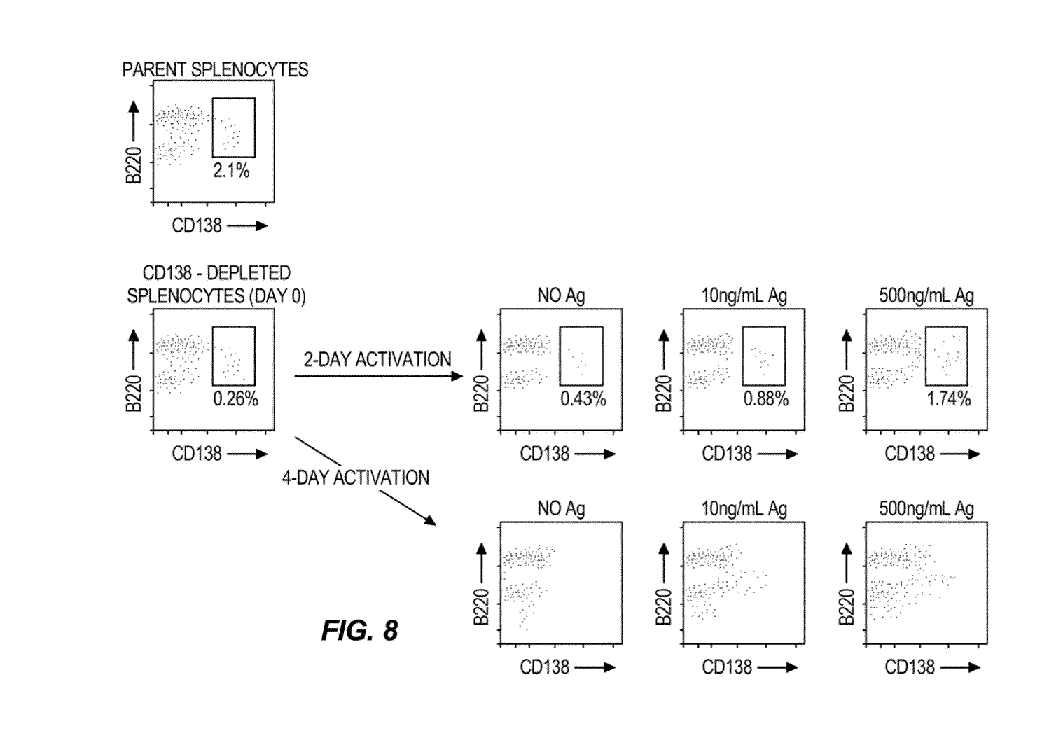

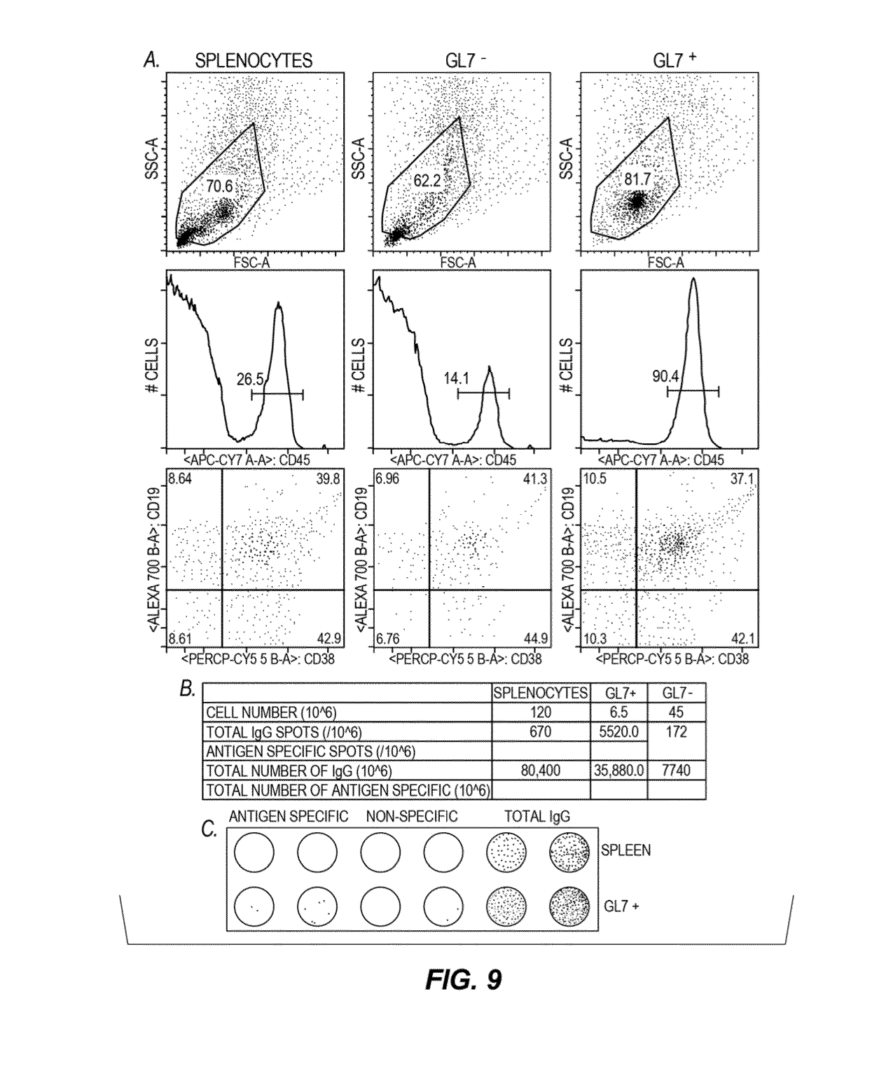

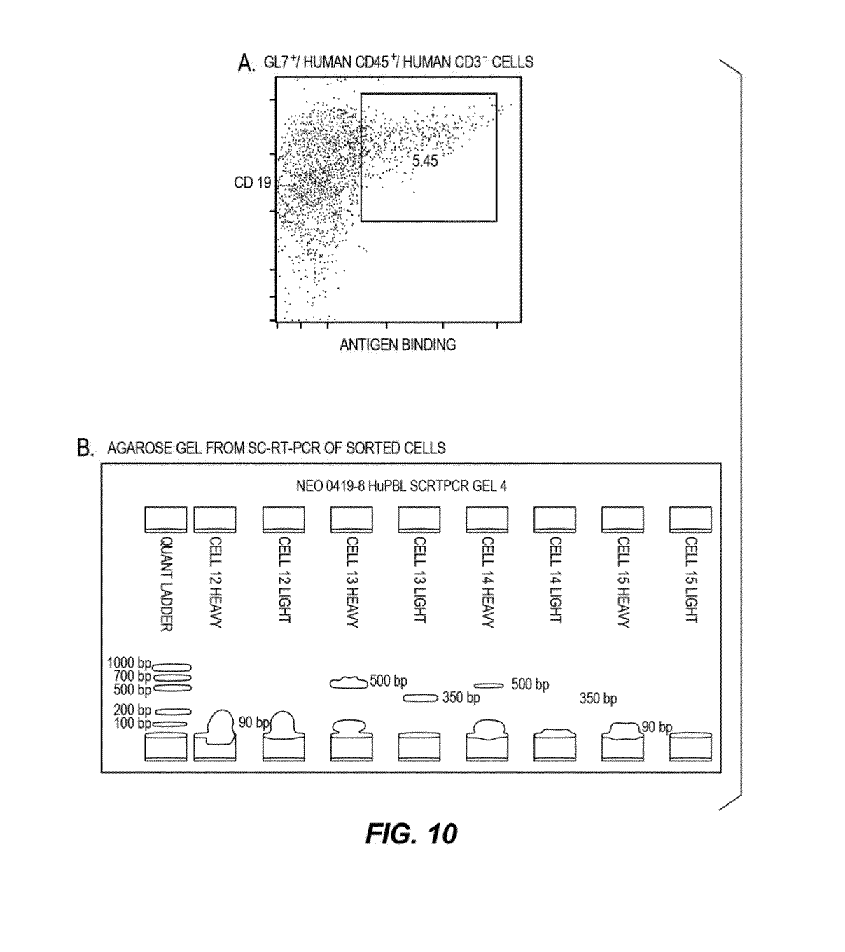

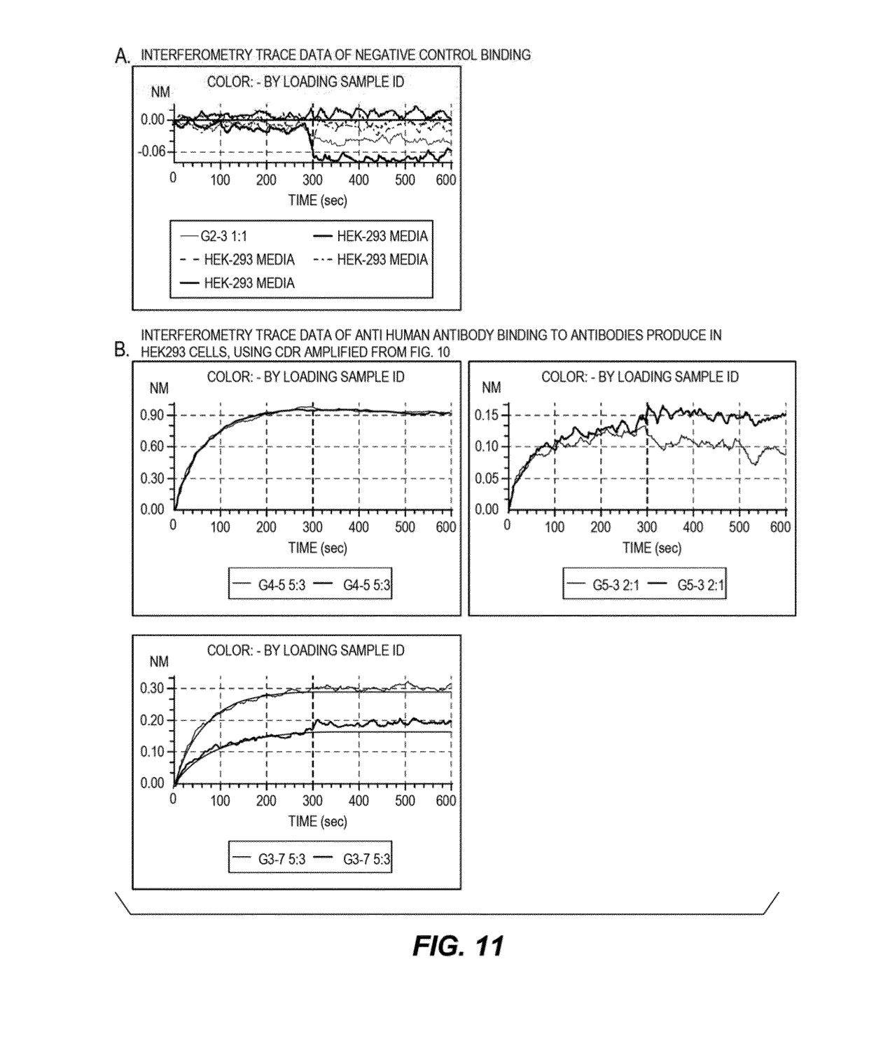

The total number of sorted, infected cells injected per recipient mouse may depend upon the population sorted, the efficiency of the sorting, and the recovery of viable, sorted cells. Typically, CD138^{high} cells represents 1-4% of the total splenocyte population (See FIG. 8A, parent splenocytes). The sorting of 10⁸ cells may theoretically yield a maximum of 1-3×10⁶ CD138^{high} cells, but the yield may be lower due to cell death during the sort. Therefore, cells may be injected based on the number of cells present in unsorted splenocytes. For example, a population of 5×10⁶ total, infected splenocytes per mouse for ascites development may be estimated to contain 5 to 15×10⁴ CD138^{high} cells. For sorted CD138^{high} cells, 5 to 15×10⁴ cells may be injected per recipient mouse depending on cell recovery.

In some processes, infected splenocytes may be injected into recipient mice with-out separating cell types. B-lymphocytes may be injected together with T-lymphocytes and monocytes/macrophages. To approximate a typical microenvironment, sorted ABL-MYC infected populations may be injected into recipient mice along with non-infected splenocytes (2.5×10⁶ per mouse). These “carrier” splenocytes may be isolated from mice immunized with a different antigen to differentiate the Ag-specificity of the infected and non-infected cells as well as to monitor viral carry-over and transformation of the carrier splenocytes. Viral carry-over can be tested by Ag-specific ELISA of developed ascites fluid using plates coated with the two different antigens.

### Example 8

### Analysis of Ascites

Ascites fluid generated may be titered for Ag-specific Ig by ELISA and tested for total Ig production. DNA may be purified from ascites cells and tested for integration of the ABL-MYC provirus into the cell genome. The cell population that is targeted by ABL-MYC may be expected to develop ascites in recipient mice within 60 days, producing plasmacytomas containing integrated ABL-MYC and secreting Ag-specific Ig.

### Example 9

### Mouse Immunization and Splenocyte Isolation

The recombinant α-subunit of E. coli RNA polymerase may be used as an exemplary antigen for primary mouse immunizations. Mice may be immunized with the α-subunit by intraperitoneal and subcutaneous injections with 50% of the protein injected at each location. Mice may be final boosted 14-28 days following the second boost. An exemplary immunization protocol is described in Table 4.

TABLE 4
Exemplary Immunization Protocol

| Immunization | | | |
|---|---|---|---|
| (per mouse) | Day | Protein | Adjuvants |
| Initial | 0 | 10 μg | Freund's Complete |
| First boost | 14 | 20 μg | Freund's Incomplete |
| Second boost | 28 | 40 μg | Freund's Incomplete |
| Final boost | 42-56 | 120 μg | PBS |

Test bleeds (75 μL taken on days 0, 28, and 42) may be used to determine the immunogenic response of the mice to the antigen by ELISA. Mice with an OD of 10 times greater than background at a test bleed 2 sera dilution of 1:1,000 may be used for final boosts and experiments.

For non-infected “carrier” splenocytes described above, mice may be immunized with GFP instead of the α subunit of E. coil RNA polymerase.

Spleens from immunized mice may be harvested 4 days after the final boost. Single-cell suspensions may be made by perfusing the spleens with 20 mL of infection media per spleen. Red blood cells may be lysed using Red Blood Cell Lysing Buffer (Sigma, St. Louis, Mo.) before resuspending in the appropriate cell media.

### Example 10

### Cell Counting

Cell recovery and viability following splenocyte isolation, cell sorting, infection, and ascites development may be assessed using an automated ViCell™ XR Cell Viability Analyzer system (Beckman Coulter, Inc., Fullerton, Calif.).

### Example 11

### ELISA

For Ag-specific ELISA, 100 ng/well antigen may be coated on a 96-well plate and then blocked with PBS containing 1% milk. Serial dilutions (1:100 to 1:12800) of mouse sera or ascites fluid may be incubated on the coated and blocked plates, and then washed with 0.1% Tween-20 in PBS (PBSST). A secondary goat anti-mouse heavy and light chain mAb conjugated to horseradish peroxidase (HRP) may be used for detection. After washings, the signal may be detected using tetramethylbenzidine liquid substrate (Sigma), stopped by the addition of 1 N H₂ SO₄, and read at 450 nm on an ELISA plate reader from Molecular Devices (Sunnyvale, Calif.).

Total IgG testing of ascites fluid may be carried out using the Easy-titer Mouse IgG Assay Kit (Pierce, Rockford, Ill.).

### Example 12

### ELISPOT

Ninety-six (96)-well PVDF plates (MSIPS4510; Millipore) may be hydrated with 70% ethanol and washed with PBS. Plates then may be coated overnight with 500 ng antigen in 100 μL per well in PBS. After a single PBS wash, plates may be blocked with cell media for at least 2 h at 37° C. before decanting. Cells may be serially diluted in media and aliquoted in duplicate onto the plate at 100 μL of cells per well. After 5 h at 37° C. in 5% CO₂. plates may be washed twice with PBS and four times with PBSST. To detect bound IgG, a 1:5000 dilution in cell media of anti-mouse IgG HRP (Sigma) may be added at 80 μL per well and incubated at 4° C. overnight. Plates may be washed 3 times with PBSST followed by 3 washes with PBS. Spots may be developed by the addition of AEC reagent (BD Biosciences) at 100 μL per well and incubated for 5-6 minutes with visual monitoring. Color development may be stopped by extensive washing with tap water. Plate backing may be removed and plates allowed to dry overnight in the dark. Spot imaging and analysis may be carried out by ZellNet Consulting (Fort Lee, N.J.).

### Example 13

### ABL-MYC Integration

DNA from cells may be isolated using the Qiagen DNeasy kit. PCR amplification may be performed with Roche's PCR Core Kit (Basel, Switzerland). Primers may extend from the 3′-end of the v-abl gene to the 5′-end the c-myc gene and include both intervening DNA sequences and the TK promoter used for murine c-MYC expression. This method may enable detection only of integrated proviral DNA where no DNA from endogenous abl or myc sequences is amplified.

### Example 14

### EL4-B5 System and Bulk Culture Optimization

The cell line EL4-B5 is a subclone of murine EL4 thymoma that is a bromo-deoxyuridine-resistant mutant. EL4-B5 is grown with B cells to activate the B cells via direct cell contact to induce proliferation, differentiation, and secretion of Ig.

Cultures may be seeded on 24-well plates in EL4-B5 media (RPMI 1640 supplemented with 100 U/mL penicillin/streptomycin, 2 mM L-glutamine, 50 μM β-mercaptoethanol, 2 mM sodium pyruvate, 10 mM HEPES and 10% FCS) along with conditioned media and/or with cytokines TLR agonists (see below). B cells (50,000) and irradiated EL4-B5 cells (250,000) may be added to each well in 1 mL. Cultures may be fed every 3 days by removing 0.5 mL of old media and adding 0.5 mL of fresh media (with conditioned media supplements).

Cultures may be harvested at 2-day intervals for 8 days. Culture media may be tested for total IgG, IgM, and IgA production to determine the total amount of Ig secreted and the extent of Ig class switching. Cells may be stained for cell determinate markers and analyzed by flow cytometry to determine the developmental state of the expanded B cells.

### Example 15

### Cytokines and Conditioned Media

Cytokines may be obtained as purified proteins or from culture supernatants of activated T cell and macrophage cultures. For human B cells, a cocktail of recombinant human IL-1β, TNFα, IL-2 and IL-10 may be sufficient for maximum B cell proliferation and Ig secretion. Conditioned media from phorbol 12-myristate 13-acetate (PMA)-stimulated EL4-B5 cells, UV stimulated PD3188 (macrophage) cells or phytohemagglutinin (PHA)/PMA-stimulated human T-cell and macrophage co-cultures may be used to support murine B cell proliferation and Ig secretion.

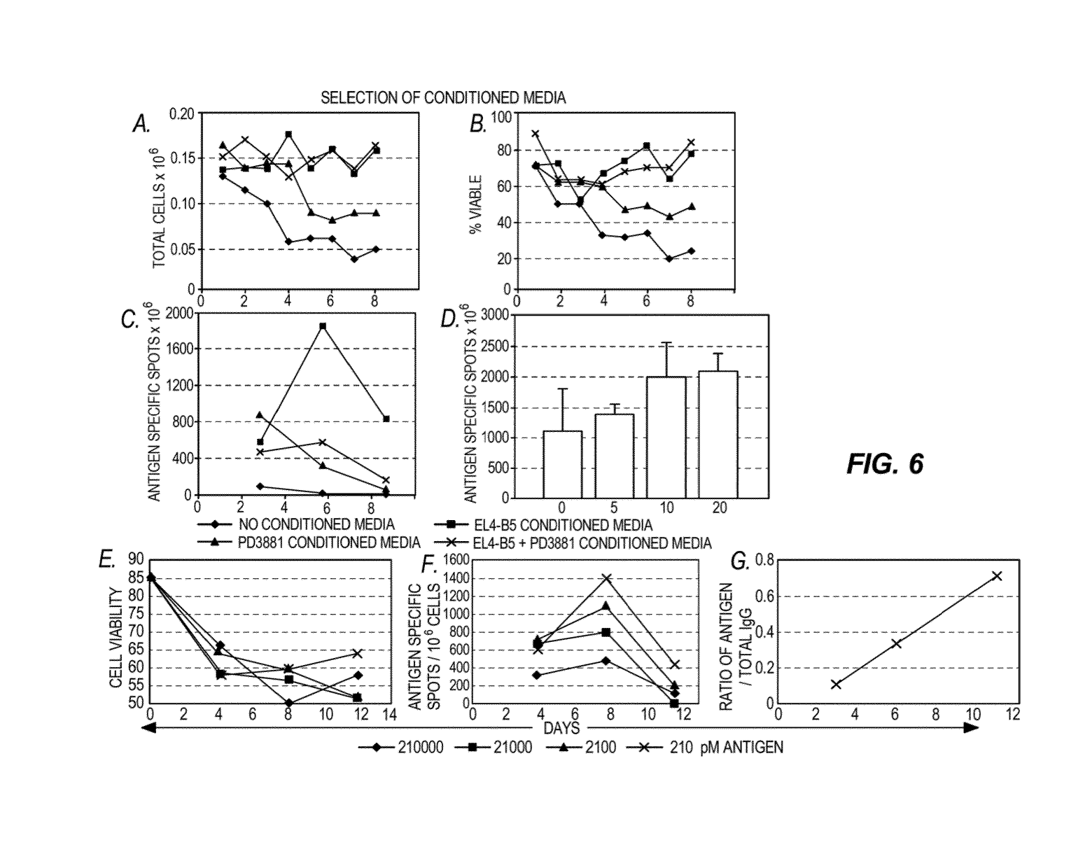

Cytokines have multiple functions, and while certain cytokines may play a role in B cell biology, they may also be detrimental. The CD4+ T cell line EL4-B5 has not been extensively studied; however, the parent line is a high IL-2 secretor that also secretes IL-6 and IFN-gamma upon stimulation. In a series of experiments to optimize the conditions (FIG. 6A-D), the conditioned medium from activated EL4-B5 cells provided better support for cell viability than the conditioned Medium from PD3881 cells. The expansion of Ag-specific Ab-secreting cells (determined by ELISpot) demonstrated that 10% EL4-B5-conditioned medium was optimal (FIG. 6D). The activated supernatant from the murine EL4-B5 cells may not perform optimally with other species, therefore the cytokine IL-21 will be added if necessary. If viability of the maturing plasmablasts is a problem the cytokines APRIL and BAFF will be examined.

### Example 16

### Single-Assay Cultures for Ag-Specific B Cell Expansion

Cultures may be propagated in 96-well plates in EL4-B5 media supplemented with the optimized conditioned media. Irradiated EL4-B5 cells (50,000) may be added to each well along with one B cell (mean per well) in 100 μL total volume. Cultures may be fed every two days by replacing 50% of the media with fresh media. Cultures may be tested at day 8 for Ag-specific Ig, at which point the cultures containing Ag-specific Ig may be infected for plasmacytoma development.

### Example 17

### B cell Selection

To increase the frequency of clonally expanded Ag-specific B cells in single cell cultures, we will use cell surface determinate labeling and magnetic depletion of non-B cell and non-Ag-specific B cells to increase the frequency of Ag-specific B cells. T-cells and monocytes/macrophages will be labeled with anti-CD3e and anti-CD11b mAbs conjugated to fluorescein isothiocyanate (FITC) (Table 3). In addition, naïve B cells may be labeled with an anti-IgD mAb conjugated to FITC. Labeled cells may be magnetically depleted using Easy-Sep Mouse FITC Selection Kit (StemCell Technologies). Confirmation of separation may be confirmed by flow cytometry and depletion conditions may be optimized, if necessary, by adjusting the amount of antibody used to label cells. The non-labeled cells may be clonally expanded as described above and the frequency of clonally expanded Ag-specific B cells may be compared to cultures of total splenocytes.

### Example 18

### Culturing and Conditioned Media

EL4-B5 cells may be grown in EL4-B5 media maintaining a cell density of less than 5×10⁵ cells per mL. To stop growth, EL4-B5 cells may be γ-irradiated at 5000 rad. Irradiated cells then may be centrifuged, resuspended in EL4-B5 media supplemented with 10% DMSO, and frozen in liquid nitrogen. Adherent P388D1 (IL-1) (ATCC, Manassas, Va.) cells may be grown in RPMI 1640 supplemented with 2 mM L-glutamine, 1.5 g/L sodium bicarbonate, 4.5 g/L glucose, 10 mM HEPES, 1 mM sodium pyruvate, and 10% FCS. Subcultures may be prepared by scraping and subcultivating at a ratio of 1:4.

Conditioned media may be prepared by culturing EL4-B5 and/or P388D1 (IL-1) cells at 5×10⁵ cells per mL in EL4-B5 media supplemented with 5 μg/mL PHA (Sigma, L-8902) and 10 ng/mL PMA (Sigma, P-8139). After no more than 36 hours, cells may be removed by centrifugation, and the supernatant may be harvested before filtering through a 0.2 pm filter. The conditioned media may be stored in aliquots at −80° C. until needed.

### Example 19

### Ig Quantification

Total IgG, IgA, and NM quantification of tissue culture supernatants may be carried out using Bethyl Laboratories (Montgomery, Tex.) Ig ELISA Quantification Kits according to the manufacture's directions.

### Example 20

### Infection and Transformation of Cultured B Cells with ABL-MYC

B cells which have been differentiated and expanded in bulk cultures are tested to determine whether they can be infected and transformed by ABL MYC. B cells may be cultured in the EL4-B5 system for the number of days required to obtain the proper level of differentiation for efficient infection and transformation.

The infected cells are injected into mice along with non-infected splenocyte carriers to ensure a suitable microenvironment for plasmacytoma development. Successful infection and transformation may be judged by one or more of: (1) the development of ascites within a suitable time frame (60 days); (2) ABL-MYC integration into DNA isolated from ascites cells; and (3) the presence of Ig in ascites fluid.

### Example 21

### ABL-MYC Transformation Efficiency

Using single-cell culturing conditions described herein, clonally expanded B cells are infected with ABL-MYC and the efficiency of transformation is determined. Transformation of B cells may be determined in at least two ways: (1) by continued in vitro culturing; and, (2) by injecting the infected cells into recipient mice. Clonally expanded, Ag-specific B cells may be infected by the addition of ABL-MYC virus directly to each well of the microplate at 50% of the total volume (i.e., 50 μL media plus 50 μL virus pool). After 4 hours at 37° C., 100 μL of media is added to each well and the plate is centrifuged at 500×g for 10 min in an Allegra 25R Beckman Centrifuge with a 96-well plate adaptor. Approximately, 100 μL of media is removed and the wash step is repeated two more times.

For in vitro transformation, infected cells are washed three times. Cells from each well are transferred and expanded into a well of a 24-well plate for plasmacytoma development. Irradiated EL4-B5 cells (250,000) are added to each well along with 1 mL of the cell media (containing optimized conditioned media as described herein). Cells are fed every 3 days by replacing 50% of the media for up to 60 days. Cultures are visually inspected every 2-3 days for expansion of transformed cells. Upon transformation, cell supernatant is tested for Ag-specific and total Ig secretion and DNA is extracted from cells to confirm ABL-MYC integration.

The infected cells also may be grown in the presence of irradiated stromal feeders such as OP-9 cells. The infected cells also may be grown in the presence of other media supplements, such as ascites fluid generated by the injection of the SP2/0 myeloma cells into pristane-primed mice or specific cytokines such as IL-6.

For injection into pristane-primed mice, the cells in each well are resuspended in PBS and transferred to a 15 mL conical tube containing 10 mL PBS. Non-infected “carrier” splenocytes (2.5×10⁶) are added to each tube immediately before centrifuging at 3000×g for 10 min. Cells are resuspended in 0.5 mL PBS and injected intraperitoneally into recipient mice for ascites development. Mice are incubated for up to 90 days and developed ascites are collected. Ascites fluid is tested for Ag-specific and total Ig. Ascites cells also is tested for DNA integration of viral ABL-MYC. Transformation efficiency is measured by the number of ABL-MYC-dependent ascites developed as a function of colonies infected and injected. Transformation efficiency is measured on a per colony basis.

### Example 22

### GL7-Enriched Splenocytes Activated In Vitro to Produce Plasmablasts in Response to Antigen

Splenocytes isolated from mice immunized with the α-subunit of E. coli RNA polymerase (Ag) were magnetically enriched for GL7-positive cells using an anti-GL7 antibody conjugated to FitC and StemCell Technologies EasySep FitC-selection kit. Selected cells (5×10⁴ cells/mL) were grown in culture in the presence of 10% EL4-B5 conditioned media along with 210 pM Ag and 25×10⁴ gamma irradiated EL4-B5 cells. FIG. 5 shows the expression profile for GL7 selected splenocytes after 2, 4, and 7 days of culture. Cells were stained with fluorophore-conjugated antibodies against the cell-determinant surface antigens CD45 (lymphocytes), B220 (B cells), CD138 (plasmablasts), CD11b (monocytes/macrophages) and CD3ε (T cells) before analysis on a Beckman LSRII benchtop cytometer. Data is not shown for CD45-negative, CD3e-positive and CD11b-positive cells. These results show that splenocytes selected for GL7 positive cells (i.e., germinal center cells) can be activated in vitro to produce plasmablasts (new targets) in an antigen-dependent manner.

### Example 21

### In Vitro Activation of GL7-Enriched Splenocytes with Antigen Results in the Secretion of Ag-Specific IgG

Parental splenocytes, and CD138 depleted enriched cells described in Example 22, and FIG. 4 were serially diluted and analyzed for antigen-specific and total IgG secretion by ELISPOT (FIG. 6). Spots developed were counted manually on a dissection microscope.

### Example 24

### In Vitro Activation of GL7-Enriched Splenocytes with Antigen Results in a Higher Probability of Developing Ag-Specific Plasmacytomas and Ascites

Cells activated in vitro (table 1) may be infected with ABL-MYC for four hours before injection into recipient mice. The mice are allowed to incubate for up to 75 days for antigen-specific plasmacytoma and ascites development. The higher ratio of antigen-specific secreting splenocytes after activation of GL7-enriched splenocytes may result in an overall increase in the number of antigens specific plasmacytomas that develop when the cells are injected intraperitoneally into BALB/c female recipient mice. The recipient mice are primed with 0.5 mL of pristane 7-10 days before receiving infected cells. Ascites fluid and cells are collected upon development for analysis.

To approximate a typical microenvironment, in vitro activated and ABL-MYC infected populations may be injected into recipient mice along with non-infected splenocytes (2.5×10⁶ per mouse). These “carrier” splenocytes may be isolated from mice immunized with a different antigen to differentiate the Ag-specificity of the infected and non-infected cells as well as to monitor viral carry-over and transformation of the carrier splenocytes. Viral carry-over can be tested by Ag-specific ELISA of developed ascites fluid using plates coated with the two different antigens.

### Example 25

### Demonstration of Antigen Specific Activation and Reactivation by ELISPOT

Splenocytes isolated from mice immunized with the α-subunit of E. coli RNA polymerase were subjected to a 4-day antigen activation after GL7, CD3, CD11b, IgD depletion, or CxCR5 enrichment. GL7-enrichment was performed using rat anti-mouse GL7 conjugated to Fluorescein isothiocyanate (FitC; BD Biosciences) and EasySep FitC Selection Kit (StemCell Technologies) according to the manufacturer's directions. GL7-enriched cells were activated for 4-days in the presence (4-day enrichment with Reactivation) or absence (4-day enrichment no Reactivation), and Non-enriched cells were activated for 4-days in the presence (4-day no enrichment with Reactivation) or absence (4-day enrichment no Reactivation). The data indicate that Ag-specific, IgG secreting cells were enriched more than 10-fold in the GL7-enriched, antigen-activated population compared to non-fractionated splenocytes (total splenocytes). In addition, enrichment plus activation leads to ˜20% more immunoglobulin secreting cells (e.g. activated B-lymphocytes) than antigen-activation in absence of enrichment (FIG. 4).

### Example 26

### Demonstration of GL7 Selection of Human Lymphocytes Harvested from a SCID Mouse Spleen Repopulated with Human Cells and Immunized to Generate a De Novo Immune Response

For generating a de novo immune response we used a SCID-huPBL engraftment model, which was coupled with Ag-pulsed DC immunizations. We used E. coli RNA polymerase CORE to perform the 25-day immunization regimen described below. (Table 5).

TABLE 5
Engraftment and immunization regimen

| Time | |
|---|---|
| Procedure | |
| Day 0 | Pre-immune bleed taken from all mice. Engraft SCID mice with |
| | huPBLs mixed with target Ag. Start 1st dendritic cell culture. |
| Day 3 | Pulse dendritic cell cultures with Ag and inject into engrafted |
| | mice. |
| Day 4 | Start 2nd dendritic cell culture. |
| Day 7 | Test bleed engrafted & immunized mice. Pulse dendritic cell |
| | cultures with Ag and inject into engrafted & immunized mice. |
| Day 14 | Test bleed engrafted & immunized mice, boost with Ag. |
| Day 21 | Test bleed engrafted & immunized mice, boost with Ag. |
| Day 24 | Take final bleed. Harvest spleen and select GL7+ fraction. |

DC culture medium is prepared using modifications to the methods of Santini et al., including 10% FBS (HyClone), 1 mM sodium pyruvate, 0.1 mM non-essential amino acids, 100 U/mL penicillin/streptomycin, and 2 mM 1-glutamine to RPMI 1640 (all reagents except FBS are GIBCO/Invitrogen). Immediately before use, 500-1000 U/mL human granulocyte-macrophage colony stimulating factor (GM-CSF) and 500-1000 U/mL interferon-0 are added. We seed DC cultures at 2×10⁶ cells/mL using CD14+ huPBLs (enriched by MACS following the manufacturer's protocol) (Miltenyi Biotec, Inc., Auburn, Calif.) and follow a 3-day culture protocol.

On day 24 the mice were sacrificed and the spleens removed. The splenocytes were obtained by pressing the spleen through a 45 micron nylon filter with the plunger from a 1 cc syringe. The mixture had the RBCs removed by lysis, The GL7+ fraction was selected by magnetic separation using the Easy-Sep anti-FITC kit. Cells from the unfractionated splenocytes, the GL7+ and the GL7− were analyzed by flow cytometry (FIG. 8A) and ELISpot (FIGS. 8B and C). The flow cytometry results indicate that the GL7+ fraction results in a population that is rich in human lymphocytes. Furthermore, the GL7+ fraction contains more than half of total IgG secreting cells determined from the whole splenocyte population. The GL7+ fraction is 5% the total cell number, meaning a 20 fold reduction in cell numbers with retention of most of the IgG secreting cells. GL7 selection captured 90% of the antigen specific cells.

The selected GL7+ human cells were cultured at 100,000 cells/well in a round bottom 96-well plate in EL-4 B5 media [RPMI 1640 supplemented with 100 U/mL penicillin/streptomycin, 2 mM L-glutamine, 50 μM 2-mercaptoethanol, 2 mM sodium pyruvate, 10 mM HEPES and 10% fetal calf serum (FCS)] with anti CD40, IL-21, ODN-2006 (CpG) linked to antigen.

### Example 27

### Selection of Antigen Specific Cells, Cell Sorting, sc-RT-PCR, Cloning and Expression of Fully Human Antibodies

Cultured GL7+ cells were run on a Becton Dickinson FACS-Diva, antigen specific B cells were selected based on CD19 expression and antigen binding. Single cells were sorted onto slides (Beckman Coulter/Advalytix AmpliGrid System), the presence of single cells confirmed by microscopy. Single cell RT-PCR was performed, generating antibody heavy and light chain. The amplified CDR regions were confirmed by sequencing. The confirmed inserts were cloned into antibody expression vectors. The successfully cloned heavy and light chain vectors were then co-transfected into HEK293 cells, the supernatant assayed for Ig production and specificity. Presence of antibody heavy and light chains recognized by an anti-human polyclonal antibody is confirmed by bio-layer interferometry (FIG. 11).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

What is claimed is:

1. A method for producing activated, antigen-specific B-lymphocytes, the method comprising:
(a) immunizing an animal;
(b) isolating lymphocytes from the animal;
(c) selecting lymphocytes positive for a cell surface antigen recognized by an anti-GL7 antibody to obtain selected lymphocytes;
(d) contacting the selected lymphocytes with an activating agent to obtain activated, antigen-specific B-lymphocytes.

2. The method of claim 1, wherein the animal is a mouse.

3. The method of claim 1, wherein the activating agent comprises the antigen.

4. The method of claim 3, wherein the activating agent further comprises antigen-specific T-cells.

5. The method of claim 3, wherein the activating agent further comprises dendritic cells.

6. The method of claim 3, wherein the activating agent further comprises macrophages.

7. The method of claim 3, wherein the activating agent further comprises an antibody.

8. The method of claim 7, wherein the antibody is an antibody against CD40.

9. The method of claim 1, wherein the selected lymphocytes and the activating agent are contacted for at least about 2 days.

10. The method of claim 1, further comprising:
(d) immortalizing the activated, antigen-specific B-lymphocytes.

11. The method of claim 10, wherein step (d) comprises transfecting the activated, antigen-specific B-lymphocytes with a viral vector that transforms the transfected cells.

12. The method of claim 10, wherein step (d) comprises fusing the activated, antigen-specific B-lymphocytes and myeloma cells.

13. A method for producing activated, antigen-specific B-lymphocytes, the method comprising:
(a) immunizing an animal, wherein the animal has been previously engrafted with a population of cells;
(c) isolating lymphocytes from the animal;
(d) selecting lymphocytes positive for a cell surface antigen recognized by an anti-GL7 antibody to obtain selected lymphocytes; and
(e) contacting the selected lympocytes with an activating agent to obtain activated, antigen-specific B-lymphocytes.

14. The method of claim 13, wherein the animal is a mouse.

15. The method of claim 13, wherein the engrafted cells are human lymphocytes depleted for CD8.

16. The method of claim 13, wherein the activating agent comprises the antigen.

17. The method of claim 16, wherein the activating agent further comprises antigen-specific T-cells, dendritic cells, macrophages, or a mixture thereof.

18. The method of claim 16, wherein the activating agent further comprises a cytokine.

19. The method of claim 16, wherein the activating agent further comprises an antibody.

20. The method of claim 16, wherein the activating agent further comprises a TLR-9 agonist.
